SEQUENCE LISTING (15 sequences)

SEQ ID NO: 1 - DNA, 20 bp, Artificial Sequence
  Description of Artificial Sequence Synthetic primer
actccagactgtccacagca

SEQ ID NO: 2 - DNA, 34 bp, Artificial Sequence
  Description of Artificial Sequence Synthetic primer
ttggggtcattttcactgggtccagcgagaaggt

SEQ ID NO: 3 - DNA, 231 bp, Artificial Sequence
  Description of Artificial Sequence Synthetic polynucleotide
actccagactgtccacagcattccgctgaccatcaataaggaagatgatgagtctccggggctctatgggtttctgaatgtcatcgtccactcagccactggatttaagcagagttcaaaagcccttcagcggccagtagcatctgactttgagcctcagggtctgagtgaagccgctcgttggaactccaaggaaaaccttctcgctggacccagtgaaaatgaccccaa

SEQ ID NO: 4 - DNA, 18 bp, Artificial Sequence
  Description of Artificial Sequence Synthetic primer
aacgatgacatgatagct

SEQ ID NO: 5 - DNA, 18 bp, Artificial Sequence
  Description of Artificial Sequence Synthetic primer
atcattacgagtgatgcg

SEQ ID NO: 6 - DNA, 18 bp, Artificial Sequence
  Description of Artificial Sequence Synthetic primer
caagaggagcgtgtccga

SEQ ID NO: 7 - DNA, 18 bp, Artificial Sequence
  Description of Artificial Sequence Synthetic primer
gatctgaatcctgcgtta

SEQ ID NO: 8 - DNA, 18 bp, Artificial Sequence
  Description of Artificial Sequence Synthetic primer
tagcagagtttcctaagg

SEQ ID NO: 9 - DNA, 18 bp, Artificial Sequence
  Description of Artificial Sequence Synthetic primer
tgcatacatagacatcca

SEQ ID NO: 10 - DNA, 18 bp, Artificial Sequence
  Description of Artificial Sequence Synthetic primer
ccacaaggtggattcgtc

SEQ ID NO: 11 - DNA, 18 bp, Artificial Sequence
  Description of Artificial Sequence Synthetic primer
tcacgcgacccacgttct

SEQ ID NO: 12 - DNA, 18 bp, Artificial Sequence
  Description of Artificial Sequence Synthetic primer
tctagtgacagtgatgcg

SEQ ID NO: 13 - DNA, 18 bp, Artificial Sequence
  Description of Artificial Sequence Synthetic primer
tgagatcattggtggatc

SEQ ID NO: 14 - DNA, 18 bp, Artificial Sequence
  Description of Artificial Sequence Synthetic primer
agtgaacgggccgttgtt

SEQ ID NO: 15 - DNA, 11 bp, Artificial Sequence
  Description of Artificial Sequence Synthetic primer
tagcagagttt

(19) United States
(12) Patent Application Publication
(10) Pub. No.: US 2021/0277458 A1
(43) Pub. Date: Sep. 9, 2021

(54) METHODS, SYSTEMS, AND APARATUS FOR NUCLEIC ACID DETECTION

(71) Applicant: Mission Bio, Inc., South San Francisco, CA (US)

(72) Inventors: Dalia Dhingra, San Francisco, CA (US); David Ruff, San Francisco, CA (US); Pedro Mendez, San Mateo, CA (US); Aik Ooi, San Mateo, CA (US)

(73) Assignee: Mission Bio, Inc., South San Francisco, CA (US)

(21) Appl. No.: 16/839,057

(22) Filed: Apr. 2, 2020

Related U.S. Application Data
(60) Provisional application No. 62/829,291, filed on Apr. 4, 2019.
(60) Provisional application No. 62/828,386, filed on Apr. 2, 2019.
(60) Provisional application No. 62/828,416, filed on Apr. 2, 2019.
(60) Provisional application No. 62/828,420, filed on Apr. 2, 2019.
(60) Provisional application No. 62/829,358, filed on Apr. 4, 2019.
(60) Provisional application No. 62/828,409, filed on Apr. 2, 2019.

(51) Int. Cl. C12Q 1/6853; C12Q 1/6869; C12N 15/62

(52) U.S. Cl. CPC C12Q 1/6853; C12Q 1/6869; C12Q 2600/158; C12Q 2600/16; C12N 15/62

(57) ABSTRACT

Provided herein are methods for detection and characterization of a target nucleic acid from a single cell. One embodiment is a method for detection of a BCR-ABL gene fusion in a nucleic acid sample from a single cell having or suspected of having a BCR-ABL fusion transcript. One preferred implementation of the invention includes providing a nucleic acid amplification primer set complementary to a target nucleic acid suspected of having a BCR-ABL fusion transcript. In some embodiments, one or both primers of the nucleic acid amplification primer set have a barcode identification sequence. Also provided are methods for the detection of an AML tumor, methods are used for the detection of a leukemia, for the detection of a myeloid leukemia, and to determine the prognosis of a patient suspected of having a BCR-ABL fusion transcript.

### FIELD

This invention relates generally to the detection and identification of target nucleic acids and mutations and allelic variants in a target nucleic acid, and more particularly to the detection and identification of target nucleic acids and mutations and allelic variants in a target nucleic acid in a single cell.

### RELATED APPLICATIONS

This application takes priority to the following U.S. Provisional Application Ser. No. 62/829,291 filed Apr. 4, 2019 and entitled ‘Method, System And Apparatus For Antibody Tag Priming And Genomic Dna Bridge’; U.S. Ser. No. 62/828,386 filed Apr. 2, 2019 and entitled ‘A Complete Solution For High Throughput Single Cell Sequencing; U.S. Ser. No. 62/828,416 filed Apr. 2, 2019 and entitled ‘Analytical Methods To Identify Tumor Heterogeneity’; U.S. Ser. No. 62/828,420 filed Apr. 2, 2019 and entitled ‘Method and Apparatus for Universal base library preparation’; and U.S. Ser. No. 62/829,358 filed Apr. 4, 2019 and entitled ‘Method and Apparatus for Fusion in DNA and RNA’, and U.S. Ser. No. 62/828,409 filed Apr. 2, 2019 and entitled ¹High Throughput Single Cell DNA Sequencing’; all incorporated by reference herein.

### BACKGROUND

There is a need for method, system and apparatus to provide high-throughput, single-cell nucleic acid detection and with pairing of genotype and phenotype. There is also need for method, system and apparatus to provide high-throughput, single-cell analyte detection and analysis that includes the detection and identification of target nucleic acids, both from DNA and RNA paired from the same single cells.

For example, the BCR-ABL gene fusion is known to be associated with the disease commonly known as Philadelphia Syndrome. The Philadelphia chromosome or Philadelphia translocation (Ph) is a specific genetic abnormality in chromosome 22 of leukemia cancer cells (particularly chronic myeloid leukemia (CML) cells). This chromosome is defective and unusually short because of reciprocal translocation, t(9;22)(q34;q11), of genetic material between chromosome 9 and chromosome 22, and contains a fusion gene called BCR-ABL1. This gene is the ABL1 gene of chromosome 9 juxtaposed onto the breakpoint cluster region BCR gene of chromosome 22, coding for a hybrid protein: a tyrosine kinase signaling protein that is “always on”, causing the cell to divide uncontrollably by interrupting the stability of the genome and impairing various signaling pathways governing the cell cycle.

The presence of this fusion transcript is a highly sensitive test for CML, since 95% of cases of CML are positive for BCR-ABL1. (Some cases are confounded by either a cryptic translocation that is invisible on G-banded chromosome preparations, or a variant translocation involving another chromosome or chromosomes as well as the long arm of chromosomes 9 and 22. Other similar but truly Ph-negative conditions are considered CML-like myeloproliferative neoplasms.) However, the presence of the Philadelphia (Ph) chromosome is not sufficiently specific to diagnose CML, since it is also found in acute lymphoblastic leukemia[4] (aka ALL, 25-30% of adult cases and 2-10% of pediatric cases) and occasionally in acute myelogenous leukemia (AML) as well as mixed-phenotype acute leukemia (MPAL). (see Wikipedia).

Better methods of identifying and characterizing allelic variants, including SNPs, mutations, fusion transcripts, gene expression, and the like are needed. The inventions described herein meet these unsolved challenges and needs.

### BRIEF SUMMARY

The inventions described and claimed herein have many attributes and embodiments including, but not limited to, those set forth or described or referenced in this Brief Summary. The inventions described and claimed herein are not limited to, or by, the features or embodiments identified in this Summary, which is included for purposes of illustration only and not restriction.

In a first aspect, embodiments of the invention are directed to the use of targeted PCR for detection and characterization of target nucleic acids from a single cell.

An exemplary embodiment of the method includes the following: selecting one or more target nucleic acid sequence of interest in an individual cell, where the target nucleic acid sequence is complementary to a nucleic acid in a cell; where the nucleic acid in the cell can be DNA or RNA; providing a sample having on or more individual single cells; encapsulating one or more individual cell in a reaction mixture comprising a protease; incubating the encapsulated cell with the protease in the drop to produce a cell lysate; providing one or more nucleic acid amplification primer sets, where each primer set is complementary to a target nucleic acid and at least one primer of a nucleic acid amplification primer set comprises a barcode sequence; performing a nucleic acid amplification reaction using the reaction mixture to form an amplification product from the nucleic acid of a single cell, where the amplification product has amplicons of one or more target nucleic acid sequence; and optionally the following, providing an affinity reagent that comprises a nucleic acid sequence complementary to the barcode sequence of one of more nucleic acid primer of a primer set, where the affinity reagent comprising said nucleic acid sequence complementary to the barcode sequence is capable of binding to a nucleic acid amplification primer set comprising a barcode sequence; contacting an affinity reagent to the amplification product comprising amplicons of one or more target nucleic acid sequence under conditions sufficient for binding of the affinity reagent to the target nucleic acid to form an affinity reagent bound target nucleic acid; and determining the identity of the target nucleic acids by sequencing the first bar code and second bar code.

In another aspect, embodiments of the invention are directed to methods and systems of PCR based detection and characterization of a target nucleic acid from a single cell. a system and method for detection of a target nucleic acid mutation or gene expression from a single cell, the method including, independent of order presented, the following steps: i) selecting one or more target nucleic acid sequence, where the target nucleic acid sequence is complementary to a nucleic acid in a cell (for example, from a population of cells including one or more cells; ii) providing a sample having on or more individual single cells; iii) encapsulating one or more individual cell in a reaction mixture comprising a protease; iv) incubating the encapsulated cell with the protease in the drop to produce a cell lysate (and optionally inactivating the protease before some other steps); v) providing one or more nucleic acid amplification primer sets, wherein each primer set is complementary to a target nucleic acid and at least one primer of a nucleic acid amplification primer set comprises a barcode sequence; vi) optionally, adding polymerases (e.g. a reverse transcriptase, or active variant thereof), primers and other necessary reaction components needed for performing reverse transcription, and performing a reverse transcription reaction from the nucleic acid of a single cell; vii) performing a nucleic acid amplification reaction to form an amplification product from the nucleic acid of a single cell, where the amplification product has amplicons of one or more target nucleic acid sequence; viii) providing an affinity reagent comprising a bead that comprises one or more nucleic acid primer comprising a barcode; ix) contacting an affinity reagent to the amplification product comprising amplicons of one or more target nucleic acid sequence under conditions sufficient for binding of the affinity reagent to the target nucleic acid to form an affinity reagent bound target nucleic acid; and x) characterizing a mutation, fusion transcript, allelic variation, or gene expression level of interest associated with the target nucleic acid by nucleic acid sequencing or other techniques.

In some implementations, solid supports, beads, and the like are coated with affinity reagents. Affinity reagents include, without limitation, antigens, antibodies or aptamers with specific binding affinity for a target molecule. The affinity reagents bind to one or more targets within the single cell entities. Affinity reagents are often detectably labeled (e.g., with a fluorophore). Affinity reagents are sometimes labeled with unique barcodes, oligonucleotide sequences, or UMIs.

In some implementations, a RT-PCR polymerase reaction is performed, for example in the reaction mixture, an addition to the reaction mixture, or added to a potion of the reaction mixture.

In some implementations, a reverse transcription reaction then amplification is performed, for example in the reaction mixture, an addition to the reaction mixture, or added to a portion of the reaction mixture.
In some implementations, a reverse transcription reaction is performed to produce a reverse transcription product.
Some implementations include performing a reverse transcription to produce a reverse transcription product before a nucleic acid amplification step.
Some implementations include performing reverse transcription on the RNA to produce a reverse transcription product and amplifying the reverse transcription product, where performing reverse transcription and amplifying occur in a single step.
Some implementations include performing a nucleic acid sequencing reaction of an amplification product.
In some implementations the affinity reagent comprises a bead or the like.

In some implementations, the method is performed by incubating the encapsulated cell in presence of protease and/or reverse transcriptase in the drop to produce cDNA and a cell lysate.

In one particular implementation, a solid support contains a plurality of affinity reagents, each specific for a different target molecule. Affinity reagents that bind a specific target molecule are collectively labeled with the same oligonucleotide sequence such that affinity molecules with different binding affinities for different targets are labeled with different oligonucleotide sequences. In this way, target molecules within a single target entity are differentially labeled in these implements.

Some variants of the above embodiments and others described herein are performed on one or more target nucleic acid sequence suspected of having a mutation, fusion transcript, or an allelic variation of interest.

One particular type of variant of interest for detection and characterization of nucleic acids and genes, including mutations, fusions and rearrangements, any nucleic acid or combination thereof. Accordingly, certain embodiments of the invention are directed to the methods for detection of a gene fusion in a nucleic acid sample from a single cell. A representative embodiment of such a method includes the following: selecting one or more target nucleic acid sequence in an individual cell; providing a sample having one or more individual single cell; encapsulating an individual cell in a drop; incubating the encapsulated cell with the protease in the drop to produce a cell lysate; providing a nucleic acid amplification primer set complementary to a target nucleic acid, where at least one primer of the nucleic acid amplification primer set includes a barcode identification sequence; performing a nucleic acid amplification reaction to form an amplification product from the nucleic acid of a single cell; and determining whether the target nucleic acid. Certain particular implementations further include providing an affinity reagent that comprises a nucleic acid sequence complementary to a barcode sequence of one of more nucleic acid primer, where the affinity reagent comprising said nucleic acid sequence complementary to the barcode sequence is capable of binding to a nucleic acid amplification primer comprising a barcode sequence; and contacting an affinity reagent to the amplification product comprising amplicons under conditions sufficient for binding of the affinity reagent to the target nucleic acid to form an affinity reagent bound target nucleic acid.

One implementation of the above method for is for the detection or characterization of a gene fusion, which may further include the nucleic acid sequencing of an amplification product or amplicon to determine whether the target nucleic acid has a fusion transcript.

One implementation includes a nucleic acid amplification primer set whose amplicon spans a splice junction.

One implementation includes a nucleic acid amplification primer set that spans a splice junction.

One implementation includes a nucleic acid amplification primer set that has a forward amplification primer having an embedded barcode identification sequence.

One implementation of the above method for detection of a gene fusion includes a nucleic acid amplification primer set that has a reverse amplification primer having an embedded barcode identification sequence.

Some implementations have more than one forward or reverse primer.

In another implementation of the above method for detection of a gene fusion method, an individual cell is encapsulated in a single drop comprising a reaction mixture in an aqueous, an aqueous emulsion in oil, or an aqueous suspension in oil.

In another implementation of the above method for detection of a gene fusion method, the reaction mixture comprises a cytolytic protease.

In another implementation of the above method for detection of a gene fusion method, the cytolytic protease comprises a proteinase K.

In another implementation of the above method for detection of a gene fusion method, the cytolytic protease is heat inactivated before or during the nucleic acid amplification reaction.

In another implementation of the above method for detection of a gene fusion method, the nucleic acid amplification reaction is the polymerase chain reaction or a known variant thereof.

One implementation of the above method for detection of a gene expression further includes the nucleic acid sequencing of an amplification product or amplicon to determine whether the target nucleic acid is present.

One implementation of the above method for detection of gene expression includes a nucleic acid amplification primer set whose amplicon spans an exon-exon boundary.

One implementation of the above method for detection of gene expression includes a nucleic acid amplification primer set that spans a splice junction.

One implementation of the above method for detection of gene expression includes a nucleic acid amplification primer set that has a forward amplification primer having an embedded barcode identification sequence.

One implementation of the above method for detection of gene expression includes a nucleic acid amplification primer set that has a reverse amplification primer having an embedded barcode identification sequence.

In another implementation of the above method for detection of a gene expression method, an individual cell is encapsulated in a single drop comprising a reaction mixture in an aqueous, an aqueous emulsion in oil, or an aqueous suspension in oil.

In another implementation of the above method for detection of a gene expression method, the reaction mixture comprises a cytolytic protease.

In another implementation of the above method for detection of a gene expression method, the cytolytic protease comprises a proteinase K.

In another implementation of the above method for detection of a gene expression method, the cytolytic protease is heat inactivated before or during the nucleic acid amplification reaction.

In another implementation of the above method for detection of a gene expression method, the nucleic acid amplification reaction is the polymerase chain reaction or a known variant thereof.

### BRIEF DESCRIPTION OF THE DRAWINGS

FIG. 1A schematically illustrates an embodiment using the Tapestri™ workflow and system for the simultaneous detection of both RNA and DNA detection on a Tapestri™ apparatus. Cells are diluted and input on the Tapestri™ Instrument where they are portioned into droplets with reagents. These reagents can include components for cell lysis, protease treatment, and reverse transcription. After the reaction in the first droplet, these encapsulated single cell lysates are input on to the Tapestri™ system for droplet merger introducing more reagents. These reagents can include materials for nucleic acid amplification and reverse transcription. FIG. 1B schematically illustrates an embodiment for the detection of BCR-ABL1 fusion transcripts and shows a fusion transcript with paired sequencing reads (read 1 and read 2). In this figure, to detect the BCR-ABL1 p210 b3a2 fusion transcript, Read 1 covers two exons in BCR. Read 2 covers the splice junction. FIG. 1C shows a microscopic image at 10× amplification of the single cell droplets after the targeted RT-PCR reaction. The targeted DNA and RNA libraries from 15%-24% of cells were sequenced on an Illumina instrument at 2×150 bp reads. Single clone cells were analyzed using Tapestri Insights v1.6.2 to genotype cells as K-562. Cells were filtered based on having 80% of the amplicons covered at greater than 10×. The results are shown in FIG. 1D. The number of RNA reads is shown on the Y axis, and the number of DNA reads is on the X-axis. RNA reads were simultaneously detected with DNA reads with a single RNA amplicon and 50 DNA amplicons. 37.5% of the cells genotypes as K-562 had 3 or more fusion reads.

FIG. 2A shows the bioanalyzer trace of a combined DNA and RNA library from a cell mixing experiment. FIG. 2B shows the fusion reads found in each genotyped cell ranked by the number of fusion reads. the results of a panel uniformity determination. With a 50:50 mixture of K-582 and Raji cells, fusion reads were only detected in K-562 cells and not in Raji cells when requiring greater than 3 fusion reads per cell. 4.8% of Raji cells had a single fusion read. 5.1% of K-562 cells had a single fusion read.

FIGS. 3A-3C shows variant detection. Panel 3A shows the results of a T-distributed stochastic neighbor embedding (t-SNE) analysis and plot. A 50:50 mix of K-562 cells and Raji cells cluster based on cell type in a t-SNA plot using 11 SNVs and insertions and/or deletions (indels). Cells genotyped as K-562 and Raji are both shown where K-562 clusters in the upper right and Raji in the bottom left. Three or more BCR-ABL1 fusion reads were found only in the K-562 cells show as triangles. In the experiments represented in FIG. 3B, a 50:50 mix ok K-562 cells and Raji cells separate into clones where Clone 1 is the expected allelic frequency for K-562 and Clone 2 is the expected allelic frequency for Raji. FIG. 3C shows the genomic variant results for nine cells where 3 or more BCR-ABL1 fusion reads were detected. For 11 SNVs, these cells with RNA reads had the expected SNV calls from DNA. Positions on the genome for allelic drop out analysis are abbreviated as ADO.

FIG. 4A shows a Bioanalyzer trace of a single cell RNA library using the Tapestri™ system. FIGS. 4B, 4C, and 4D shows images from the Integrative Genomics Viewer from the Broad Institute where each panel are the paired reads from single cells. The reads cross from exon to exon demonstrating they are from RNA molecules captured by these nucleic acid amplification primer set that do not contain introns. The reads that cross exon-intron boundaries could be from DNA molecules targeted by the same primers. FIG. 4B shows the GUSB gene. FIG. 4C shows the ITGAM gene and FIG. 4D shows the NFKBIA gene.

FIGS. 5A-5B schematically illustrate an embodiment using the Tapestri™ workflow and system for the simultaneous detection of both RNA and DNA detection where a single library is produced. FIG. 5A shows the reverse transcription reaction and protease treatment in the first droplet on the Tapestri™ platform while FIG. 5B shows the targeted amplification including the attachment of the cell barcode.

FIG. 6 shows a Bioanalyzer trace of libraries where both DNA and RNA were targeted in a three cell line mixture, K-562, TOM-1, and Raji. BCR-ABL1 was targeted along with 128 DNA amplicons with relevant mutations in acute myeloid leukemia. K-562 is positive for the BCR-ABL1 p210 b3a2 fusion transcript. TOM-1 is positive for the BCR-ABL1 p190 fusion transcript. Raji has no BCR-ABL1 fusion transcripts.

FIGS. 7A-7C present the performance of the DNA panel and the RNA fusion panel from the libraries shown in FIG. 6. FIG. 7A describes DNA panel performance with the total number of cells, the sequencing reads on target, and panel uniformity. FIG. 7B shows the number of cells genotyped from 3 single nucleotide variants found in the DNA panel data and the number of cells with 1, 3, 5 or 10 or more fusion sequencing reads. FIG. 7C shows the calculations of sensitivity and specificity when 5 fusion reads are required for a positive call. The sensitivity for p210 b3-a2 detection was 92.0% and for p190 e1-a2 detection was 69.2%. Both had specificities of greater than 98%. Raji had no fusions called

FIGS. 8A-8D show results from a combined DNA and RNA library from a 4 cell line mixture. K-562, positive for BCR-ABL1 p210 b3a2, KCL-22, positive for BCR-ABL1 p210 b2a2, TOM-1, positive for BCR-ABL1 p190, and KG-1, negative for all BCR-ABL1 fusion transcripts were used. Primers targeting these three BCR-ABL1 fusions were used with a 128 plex acute myeloid leukemia DNA panel. FIG. 8A shows the 1311 genotyped cells and the number of cells with 5 or more fusion sequencing reads. FIG. 8B shows the sensitivity and specificity calculations for each fusion transcript when requiring 5 or more fusion sequencing reads per cell. FIG. 8C increases the fusion sequencing reads required for the same 1311 genotyped cells to 20 or more. FIG. 8D shows the sensitivity and improved specificities calculated for the three fusion transcripts.

FIGS. 9A-9B schematically illustrate an embodiment using the Tapestri™ workflow and system for the simultaneous detection of both RNA and DNA detection where libraries from targeted RNA and targeted DNA can be separated. FIG. 9A shows the reverse transcription reaction and protease treatment in the first droplet on the Tapestri™ platform while FIG. 9B shows the targeted amplification including the attachment of the cell barcode. The RNA and DNA amplicons from this workflow can be separated into different sequencing libraries.

FIGS. 10A-10B shows results from single cells where a 110 plex RNA library and an 88 plex DNA library are made from the same single cells. FIG. 10A describes DNA panel performance with the total number of cells, the sequencing reads on target, and panel uniformity when reverse transcription primers had an annealing temperature of 63 C. FIG. 10B describes DNA panel performance with the total number of cells, the sequencing reads on target, and panel uniformity when reverse transcription primers had an annealing temperature of 45 C.

FIGS. 11A-11D shows the corresponding RNA library performance from the same single cells as described in FIGS. 10A-10B. FIG. 11A shows the cells from the 63 C reverse transcription primers clustered by umap based on RNA sequencing results but colored by their genotyping data from the DNA libraries. The Jurkat cells are colored red, the Y79 cells are colored orange, the KG1 cells are colored blue. Cells classified as a mixture of 2 or more cells are colored purple and cells that could not be genotyped by the genotyping data are colored gray. FIG. 11B shows violin plots of the number of genes found in each single cell separated out by cell type, determined from the DNA panel data using the 63 C reverse transcription primers. FIG. 11C are the umap clusters from the 45 C reverse transcription primer single cells. The Jurkat cells are colored red, the Y79 cells are colored orange, the KG1 cells are colored blue. Cells classified as a mixture of 2 or more cells are colored purple and cells that could not be genotyped by the genotyping data are colored gray. FIG. 11D shows violin plots of the number of genes found in each single cell separated out by cell type, determined from the DNA panel data using the 45 C reverse transcription primers.

FIG. 12A shows an image of the PTEN gene from the Integrative Genomics Viewer from the Broad Institute where each panel are the paired reads from RNA libraries made without protease (top panel) and with protease (bottom panel). The reads cross from exon to exon demonstrating they are from RNA molecules captured by these targeted primers that do not contain introns. The reads that cross exon-intron boundaries could be from DNA molecules targeted by the same primers. FIG. 12B shows the genes per cell for the 354 cells genotyped by DNA.

FIG. 13A shows the DNA panel performance for the same 354 cells whose RNA panel performance was shown in FIG. 12. The 354 cells were identified using the number of reads from the dna panel, the cell uniformity, and the amplicon uniformity, seen in FIG. 13A. FIG. 13B is a heatmap of the single nucleotide variants from the DNA library of these 354 cells. The heatmap shows 2 clusters corresponding with 2 cell types used in this experiment. FIG. 13C is a cluster of the cells based on the single nucleotide variants.

FIG. 14 is a schematic illustration of an exemplary embodiment of a bead with an externally-linked primer.

FIG. 15 is an illustration of an exemplary application of an externally-linked primer to bead

### DETAILED DESCRIPTION

Various aspects of the invention will now be described with reference to the following section which will be understood to be provided by way of illustration only and not to constitute a limitation on the scope of the invention.

“Complementarity” refers to the ability of a nucleic acid to form hydrogen bond(s) or hybridize with another nucleic acid sequence by either traditional Watson-Crick or other non-traditional types. As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA. See e.g. Ausubel, et al., Current Protocols In Molecular Biology, John Wiley & Sons, New York, N.Y., 1993. If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are “substantially complementary” to each other when a sufficient number of corresponding positions in each molecule are occupied by nucleotides that can hybridize or anneal with each other in order to affect the desired process. A complementary sequence is a sequence capable of annealing under stringent conditions to provide a 3-terminal serving as the origin of synthesis of complementary chain.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, Md.). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

The terms “amplify”, “amplifying”, “amplification reaction” and their variants, refer generally to any action or process whereby at least a portion of a nucleic acid molecule (referred to as a template nucleic acid molecule) is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule. Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes the simultaneous amplification of a plurality of target sequences in a single amplification reaction. At least some of the target sequences can be situated, on the same nucleic acid molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further including any of the amplification processes known to one of ordinary skill in the art. In some embodiments, the amplification reaction includes polymerase chain reaction (PCR). In the present invention, the terms “synthesis” and “amplification” of nucleic acid are used. The synthesis of nucleic acid in the present invention means the elongation or extension of nucleic acid from an oligonucleotide serving as the origin of synthesis. If not only this synthesis but also the formation of other nucleic acid and the elongation or extension reaction of this formed nucleic acid occur continuously, a series of these reactions is comprehensively called amplification. The polynucleic acid produced by the amplification technology employed is generically referred to as an “amplicon” or “amplification product.”

A number of nucleic acid polymerases can be used in the amplification reactions utilized in certain embodiments provided herein, including any enzyme that can catalyze the polymerization of nucleotides (including analogs thereof) into a nucleic acid strand. Such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also includes fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer-based polymerase that optionally can be reactivated.

A number of nucleic acid reverse transcriptases described herein or known in the art can be used in the extension reactions utilized in certain embodiments provided herein, including any enzyme that can catalyze the polymerization of nucleotides (including analogs thereof) into a nucleic acid strand. Such nucleotide polymerization can occur in a template-dependent fashion.

“Target nucleic acids' may include any type or combination of nucleic acid, naturally occurring or modified, including but not limited to DNA (e.g., genomic DNA (gDNA), nDNA, cDNA, mitochondrial DNA, bacterial DNA, viral DNA, etc.) and RNA (e.g. mRNA, non-coding RNA's, mitochondrial RNA, bacterial RNA, viral RNA, etc., rRNA, tRNA, tmRNA, snRNA, snoRNA, SmY, scaRNA, gRNAmiRNA, siRNA, etc.)

The terms “target primer” or “target-specific primer” and variations thereof refer to primers that are complementary to a binding site sequence. Target primers are generally a single stranded or double-stranded polynucleotide, typically an oligonucleotide, that includes at least one sequence that is at least partially complementary to a target nucleic acid sequence.

“Forward primer binding site” and “reverse primer binding site” refers to the regions on the template DNA and/or the amplicon to which the forward and reverse primers bind. The primers act to delimit the region of the original template polynucleotide which is exponentially amplified during amplification. In some embodiments, additional primers may bind to the region 5′ of the forward primer and/or reverse primers, which may bind a reverse transcription binding site. Where such additional primers are used, the forward primer binding site and/or the reverse primer binding site may encompass the binding regions of these additional primers as well as the binding regions of the primers themselves. For example, in some embodiments, the method may use one or more additional primers which bind to a region that lies 5′ of the forward and/or reverse primer binding region. Such a method was disclosed, for example, in WO0028082 which discloses the use of “displacement primers” or “outer primers”.

A ‘barcode’ nucleic acid identification sequence can be incorporated into a nucleic acid primer or linked to a primer to enable independent sequencing and identification to be associated with one another via a barcode which relates information and identification that originated from molecules that existed within the same sample. There are numerous techniques that can be used to attach barcodes to the nucleic acids within a discrete entity. For example, the target nucleic acids may or may not be first amplified and fragmented into shorter pieces. The molecules can be combined with discrete entities, e.g., droplets, containing the barcodes. The barcodes can then be attached to the molecules using, for example, splicing by overlap extension. In this approach, the initial target molecules can have “adaptor” sequences added, which are molecules of a known sequence to which primers can be synthesized. When combined with the barcodes, primers can be used that are complementary to the adaptor sequences and the barcode sequences, such that the product amplicons of both target nucleic acids and barcodes can anneal to one another and, via an extension reaction such as DNA polymerization, be extended onto one another, generating a double-stranded product including the target nucleic acids attached to the barcode sequence. Alternatively, the primers that amplify that target can themselves be barcoded so that, upon annealing and extending onto the target, the amplicon produced has the barcode sequence incorporated into it. This can be applied with a number of amplification strategies, including specific amplification with PCR or non-specific amplification with, for example, MDA. An alternative enzymatic reaction that can be used to attach barcodes to nucleic acids is ligation, including blunt or sticky end ligation. In this approach, the DNA barcodes are incubated with the nucleic acid targets and ligase enzyme, resulting in the ligation of the barcode to the targets. The ends of the nucleic acids can be modified as needed for ligation by a number of techniques, including by using adaptors introduced with ligase or fragments to enable greater control over the number of barcodes added to the end of the molecule.

A barcode sequence can additionally be incorporated into microfluidic beads to decorate the bead with identical sequence tags. Such tagged beads can be inserted into microfluidic droplets and via droplet PCR amplification, tag each target amplicon with the unique bead barcode. Such barcodes can be used to identify specific droplets upon a population of amplicons originated from. This scheme can be utilized when combining a microfluidic droplet containing single individual cell with another microfluidic droplet containing a tagged bead. Upon collection and combination of many microfluidic droplets, amplicon sequencing results allow for assignment of each product to unique microfluidic droplets. In a typical implementation, we use barcodes on the Mission Bio Tapestri™ beads to tag and then later identify each droplet's amplicon content. The use of barcodes is described in U.S. patent application Ser. No. 15/940,850 filed Mar. 29, 2018 by Abate, A. et al., entitled ‘Sequencing of Nucleic Acids via Barcoding in Discrete Entities’, incorporated by reference herein.

In some embodiments, it may be advantageous to introduce barcodes into discrete entities, e.g., microdroplets, on the surface of a bead, such as a solid polymer bead or a hydrogel bead. These beads can be synthesized using a variety of techniques. For example, using a mix-split technique, beads with many copies of the same, random barcode sequence can be synthesized. This can be accomplished by, for example, creating a plurality of beads including sites on which DNA can be synthesized. The beads can be divided into four collections and each mixed with a buffer that will add a base to it, such as an A, T, G, or C. By dividing the population into four subpopulations, each subpopulation can have one of the bases added to its surface. This reaction can be accomplished in such a way that only a single base is added and no further bases are added. The beads from all four subpopulations can be combined and mixed together, and divided into four populations a second time. In this division step, the beads from the previous four populations may be mixed together randomly. They can then be added to the four different solutions, adding another, random base on the surface of each bead. This process can be repeated to generate sequences on the surface of the bead of a length approximately equal to the number of times that the population is split and mixed. If this was done 10 times, for example, the result would be a population of beads in which each bead has many copies of the same random 10-base sequence synthesized on its surface. The sequence on each bead would be determined by the particular sequence of reactors it ended up in through each mix-spit cycle.

A barcode may further comprise a ‘unique identification sequence’ (UMI). A UMI is a nucleic acid having a sequence which can be used to identify and/or distinguish one or more first molecules to which the UMI is conjugated from one or more second molecules. UMIs are typically short, e.g., about 5 to 20 bases in length, and may be conjugated to one or more target molecules of interest or amplification products thereof. UMIs may be single or double stranded. In some embodiments, both a nucleic acid barcode sequence and a UMI are incorporated into a nucleic acid target molecule or an amplification product thereof. Generally, a UMI is used to distinguish between molecules of a similar type within a population or group, whereas a nucleic acid barcode sequence is used to distinguish between populations or groups of molecules. In some embodiments, where both a UMI and a nucleic acid barcode sequence are utilized, the UMI is shorter in sequence length than the nucleic acid barcode sequence.

The terms “identity” and “identical” and their variants, as used herein, when used in reference to two or more nucleic acid sequences, refer to similarity in sequence of the two or more sequences (e.g., nucleotide or polypeptide sequences). In the context of two or more homologous sequences, the percent identity or homology of the sequences or subsequences thereof indicates the percentage of all monomeric units (e.g., nucleotides or amino acids) that are the same (i.e., about 70% identity, preferably 75%, 80%, 85%, 90%, 95%, 97%, 98% or 99% identity). The percent identity can be over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 85% identity at the amino acid level or at the nucleotide level. Preferably, the identity exists over a region that is at least about 25, 50, or 100 residues in length, or across the entire length of at least one compared sequence. A typical algorithm for determining percent sequence identity and sequence similarity are the BLAST and BLAST 2.0 algorithms, which are described in Altschul et al, Nuc. Acids Res. 25:3389-3402 (1977). Other methods include the algorithms of Smith & Waterman, Adv. Appl. Math. 2:482 (1981), and Needleman & Wunsch, J. Mol. Biol. 48:443 (1970), etc. Another indication that two nucleic acid sequences are substantially identical is that the two molecules or their complements hybridize to each other under stringent hybridization conditions.

The terms “nucleic acid,” “polynucleotides,” and “oligonucleotides” refers to biopolymers of nucleotides and, unless the context indicates otherwise, includes modified and unmodified nucleotides, and both DNA and RNA, and modified nucleic acid backbones. For example, in certain embodiments, the nucleic acid is a peptide nucleic acid (PNA) or a locked nucleic acid (LNA). Typically, the methods as described herein are performed using DNA as the nucleic acid template for amplification. However, nucleic acid whose nucleotide is replaced by an artificial derivative or modified nucleic acid from natural DNA or RNA is also included in the nucleic acid of the present invention insofar as it functions as a template for synthesis of complementary chain. The nucleic acid of the present invention is generally contained in a biological sample. The biological sample includes animal, plant or microbial tissues, cells, cultures and excretions, or extracts therefrom. In certain aspects, the biological sample includes intracellular parasitic genomic DNA or RNA such as virus or mycoplasma. The nucleic acid may be derived from nucleic acid contained in said biological sample. For example, genomic DNA, or cDNA synthesized from mRNA, or nucleic acid amplified on the basis of nucleic acid derived from the biological sample, are preferably used in the described methods. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes thymidine, and “U’ denotes deoxyuridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

A template nucleic acid is a nucleic acid serving as a template for synthesizing a complementary chain in a nucleic acid amplification technique. A complementary chain having a nucleotide sequence complementary to the template has a meaning as a chain corresponding to the template, but the relationship between the two is merely relative. That is, according to the methods described herein a chain synthesized as the complementary chain can function again as a template. That is, the complementary chain can become a template. In certain embodiments, the template is derived from a biological sample, e.g., plant, animal, virus, micro-organism, bacteria, fungus, etc. In certain embodiments, the animal is a mammal, e.g., a human patient. A template nucleic acid typically comprises one or more target nucleic acid. A target nucleic acid in exemplary embodiments may comprise any single or double-stranded nucleic acid sequence that can be amplified or synthesized according to the disclosure, including any nucleic acid sequence suspected or expected to be present in a sample.

Primers and oligonucleotides used in embodiments herein comprise nucleotides. A nucleotide comprises any compound, including without limitation any naturally occurring nucleotide or analog thereof, which can bind selectively to, or can be polymerized by, a polymerase or extended by a reverse transcriptase. Typically, but not necessarily, selective binding of the nucleotide to the polymerase or reverse transcriptase is followed by extension of the nucleotide into a nucleic acid strand by the polymerase or reverse transcriptase; occasionally however the nucleotide may dissociate from the polymerase without becoming incorporated into the nucleic acid strand, an event referred to herein as a “non-productive” event. Such nucleotides include not only naturally occurring nucleotides but also any analogs, regardless of their structure, that can bind selectively to, or can be polymerized by, a polymerase or extended by a reverse transcriptase. While naturally occurring nucleotides typically comprise base, sugar and phosphate moieties, the nucleotides of the present disclosure can include compounds lacking any one, some or all of such moieties. For example, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain can have side groups having O, BH3, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281.

In some embodiments, the nucleotide comprises a label and referred to herein as a “labeled nucleotide”; the label of the labeled nucleotide is referred to herein as a “nucleotide label”. In some embodiments, the label can be in the form of a fluorescent moiety (e.g. dye), luminescent moiety, or the like attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g. α-thio-nucleotide 5′-triphosphates. For a review of nucleic acid chemistry, see: Shabarova, Z. and Bogdanov, A. Advanced Organic Chemistry of Nucleic Acids, VCH, New York, 1994.

Any nucleic acid amplification method may be utilized, such as a PCR-based assay, e.g., quantitative PCR (qPCR), or an isothermal amplification may be used to detect the presence of certain nucleic acids, e.g., genes, of interest, present in discrete entities or one or more components thereof, e.g., cells encapsulated therein. Such assays can be applied to discrete entities within a microfluidic device or a portion thereof or any other suitable location. The conditions of such amplification or PCR-based assays may include detecting nucleic acid amplification over time and may vary in one or more ways.

Any nucleic acid extension method may be utilized, such as a reverse transcription reaction may be used to detect the presence of certain nucleic acids, e.g., genes, of interest, present in discrete entities or one or more components thereof, e.g., cells encapsulated therein. Such assays can be applied to discrete entities within a microfluidic device or a portion thereof or any other suitable location. The conditions of such extension or reverse transcription assays may include detecting nucleic acid amplification over time and may vary in one or more ways.

The number of amplification/PCR primers that may be added to a microdroplet may vary. The number of amplification or PCR primers that may be added to a microdroplet may range from about 1 to about 500 or more, e.g., about 2 to 100 primers, about 2 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more.

The number of reverse transcription primers that may be added to a microdroplet may vary. The number of reverse transcription primers that may be added to a microdroplet may range from about 1 to about 500 or more, e.g., about 2 to 100 primers, about 2 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more.

One or both primers of a primer set may comprise a barcode sequence. In some embodiments, one or both primers comprise a barcode sequence and a unique molecular identifier (UMI). In some embodiments, where both a UMI and a nucleic acid barcode sequence are utilized, the UMI is incorporated into the target nucleic acid or an amplification product thereof prior to the incorporation of the nucleic acid barcode sequence. In some embodiments, where both a UMI and a nucleic acid barcode sequence are utilized, the nucleic acid barcode sequence is incorporated into the UMI or an amplification product thereof subsequent to the incorporation of the UMI into a target nucleic acid or an amplification product thereof.

One or multiple primer of a primer set may also be attached or conjugated to an affinity reagent. In some embodiments, individual cells, for example, are isolated in discrete entities, e.g., droplets. These cells may be lysed and their nucleic acids barcoded. This process can be performed on a large number of single cells in discrete entities with unique barcode sequences enabling subsequent deconvolution of mixed sequence reads by barcode to obtain single cell information. This approach provides a way to group together nucleic acids originating from large numbers of single cells. Additionally, affinity reagents such as antibodies can be conjugated with nucleic acid labels, e.g., oligonucleotides including barcodes, which can be used to identify antibody type, e.g., the target specificity of an antibody. These reagents can then be used to bind to the proteins within or on cells, thereby associating the nucleic acids carried by the affinity reagents to the cells to which they are bound. These cells can then be processed through a barcoding workflow as described herein to attach barcodes to the nucleic acid labels on the affinity reagents. Techniques of library preparation, sequencing, and bioinformatics may then be used to group the sequences according to cell/discrete entity barcodes. Any suitable affinity reagent that can bind to or recognize a biological sample or portion or component thereof, such as a protein, a molecule, or complexes thereof, may be utilized in connection with these methods. The affinity reagents may be labeled with nucleic acid sequences that relates their identity, e.g., the target specificity of the antibodies, permitting their detection and quantitation using the barcoding and sequencing methods described herein. Exemplary affinity reagents can include, for example, antibodies, antibody fragments, Fabs, scFvs, peptides, drugs, etc. or combinations thereof. The affinity reagents, e.g., antibodies, can be expressed by one or more organisms or provided using a biological synthesis technique, such as phage, mRNA, or ribosome display. The affinity reagents may also be generated via chemical or biochemical means, such as by chemical linkage using N-Hydroxysuccinimide (NETS), click chemistry, or streptavidin-biotin interaction, for example. The oligo-affinity reagent conjugates can also be generated by attaching oligos to affinity reagents and hybridizing, ligating, and/or extending via polymerase, etc., additional oligos to the previously conjugated oligos. An advantage of affinity reagent labeling with nucleic acids is that it permits highly multiplexed analysis of biological samples. For example, large mixtures of antibodies or binding reagents recognizing a variety of targets in a sample can be mixed together, each labeled with its own nucleic acid sequence. This cocktail can then be reacted to the sample and subjected to a barcoding workflow as described herein to recover information about which reagents bound, their quantity, and how this varies among the different entities in the sample, such as among single cells. The above approach can be applied to a variety of molecular targets, including samples including one or more of cells, peptides, proteins, macromolecules, macromolecular complexes, etc. The sample can be subjected to conventional processing for analysis, such as fixation and permeabilization, aiding binding of the affinity reagents. To obtain highly accurate quantitation, the unique molecular identifier (UMI) techniques described herein can also be used so that affinity reagent molecules are counted accurately. This can be accomplished in a number of ways, including by synthesizing UMIs onto the labels attached to each affinity reagent before, during, or after conjugation, or by attaching the UMIs microfluidically when the reagents are used. Similar methods of generating the barcodes, for example, using combinatorial barcode techniques as applied to single cell sequencing and described herein, are applicable to the affinity reagent technique. These techniques enable the analysis of proteins and/or epitopes in a variety of biological samples to perform, for example, mapping of epitopes or post translational modifications in proteins and other entities or performing single cell proteomics. For example, using the methods described herein, it is possible to generate a library of labeled affinity reagents that detect an epitope in all proteins in the proteome of an organism, label those epitopes with the reagents, and apply the barcoding and sequencing techniques described herein to detect and accurately quantitate the labels associated with these epitopes.

Primers may contain primers for one or more nucleic acid of interest, e.g. one or more genes of interest. The number of primers for genes of interest that are added may be from about one to 500, e.g., about 1 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more. Primers and/or reagents may be added to a discrete entity, e.g., a microdroplet, in one step, or in more than one step. For instance, the primers may be added in two or more steps, three or more steps, four or more steps, or five or more steps. Regardless of whether the primers are added in one step or in more than one step, they may be added after the addition of a lysing agent, prior to the addition of a lysing agent, or concomitantly with the addition of a lysing agent. When added before or after the addition of a lysing agent, the PCR primers may be added in a separate step from the addition of a lysing agent. In some embodiments, the discrete entity, e.g., a microdroplet, may be subjected to a dilution step and/or enzyme inactivation step prior to the addition of the PCR reagents. Exemplary embodiments of such methods are described in PCT Publication No. WO 2014/028378, the disclosure of which is incorporated by reference herein in its entirety and for all purposes.

A primer set for the amplification of a target nucleic acid typically includes a forward primer and a reverse primer that are complementary to a target nucleic acid or the complement thereof. In some embodiments, amplification can be performed using multiple target-specific primer pairs in a single amplification reaction, wherein each primer pair includes a forward target-specific primer and a reverse target-specific primer, where each includes at least one sequence that substantially complementary or substantially identical to a corresponding target sequence in the sample, and each primer pair having a different corresponding target sequence. Accordingly, certain methods herein are used to detect or identify multiple target sequences from a single cell sample.

In another aspect, universal base nucleotides (universal bases) can be incorporated into sites in an amplicon that can be used as a unique identifier in the nucleic acid. Two implementations to perform targeted library preparation with the ability to count unique molecules with nucleic acids as the starting material are provided. Various embodiments can be used with DNA, RNA, or DNA combined with RNA as the target molecules. For our first approach, we have designed gene specific primers with tails where a universal base is incorporated within the gene specific sequence. These universal bases still allow stable hybridization to the target nucleic acid with the remaining natural bases forcing the required specificity. These gene specific primers can be extended with primer extension or reverse transcription. During the second cycle of amplification (e.g. PCR) or second strand synthesis, the polymerase or reverse transcriptase incorporates random at these universal base sites. Each of these random bases form a unique identifier that can be amplified in these embodiments. This approach can be performed with single cells or bulk samples. For single cells, after the second copy is synthesized resulting in a targeted molecule with tails on both ends, the emulsion can be broken and for either type of sample, the gene specific primers are removed. This is followed by bulk library PCR using these tail sequences to either amplify the target molecules or to add on complete sequencing adaptors. During analysis, the gene specific primer sequences are known and single nucleotide variations would not be attributed to the sample. The sites where a universal base has been incorporated could be used to distinguish each original molecule.

For our second approach, our gene specific primers are followed by a single sequence that comprises a chain of universal bases then the tail sequence. These universal bases contribute to the hybridization of the gene specific primer to the target. During the second cycle of PCR or second strand synthesis, the polymerase or reverse transcriptase incorporates random bases pairing with these universal bases creating a unique identifier for that molecule. For single cells, at this point the emulsion would be broken and for single cell and bulk samples, all gene specific primers removed. Now each of these unique identifiers can be amplified in bulk during library PCR using the tail sequences to either amplify the target molecules or to add on complete sequencing adaptors. The chain of bases preceding the gene specific primer could then be used to identify each original molecule.

In some embodiments, this second approach can also be used without gene specific primers for DNA where ligation can be performed with a molecule containing a known short sequence, a chain of universal bases, and a tail sequence. Tail sequences can be used for amplification, using a gene specific primer for one primer or solely using tail sequences. Universal bases or universal-like bases such as deoxyinosine and derivatives thereof or nitroazole analogues (e.g. 5-nitroindole) can be incorporated into primers and amplification products. Many universal bases, including those known or described in the literature, can be used for either of these approaches or other embodiments described herein (see Loakes, D., The applications of universal base analogues, Nucleic Acids Research, Vol. 29, No. 12, 2437-2447 (2001), incorporated by reference herein).

An exemplary embodiment is a system and method for detection of a target nucleic acid from a single cell, the method including, independent of order presented, the following steps: selecting one or more target nucleic acid sequence of interest in an individual cell, where the target nucleic acid sequence is complementary to a nucleic acid in a cell; providing a sample having on or more individual single cells; encapsulating one or more individual cell in a reaction mixture comprising a protease; incubating the encapsulated cell with the protease in the drop to produce a cell lysate; providing one or more nucleic acid amplification primer sets, wherein each primer set is complementary to a target nucleic acid and at least one primer of a nucleic acid amplification primer set comprises a barcode sequence; providing one or more universal bases in an nucleic acid amplification reaction mixture; performing a nucleic acid amplification reaction using the reaction mixture comprising the universal bases to form an amplification product from the nucleic acid of a single cell, where the amplification product has amplicons of one or more target nucleic acid sequence; and optionally the following, providing an affinity reagent that comprises a nucleic acid sequence complementary to the barcode sequence of one of more nucleic acid primer of a primer set, where the affinity reagent comprising said nucleic acid sequence complementary to the barcode sequence is capable of binding to a nucleic acid amplification primer set comprising a barcode sequence; contacting an affinity reagent to the amplification product comprising amplicons of one or more target nucleic acid sequence under conditions sufficient for binding of the affinity reagent to the target nucleic acid to form an affinity reagent bound target nucleic acid; and determining the identity of the target nucleic acids by sequencing the first bar code and second bar code.

### Identification of Target Nucleic Acids, SNPs, Translocations, Polymorphisms, Allelic Variants and Other Mutations Using Methods and Systems for High Throughput Single Cell Sequencing

A fundamental challenge in precision medicine has been improving the understanding of cancer heterogeneity and clonal evolution, which has major implications in targeted therapy selection and disease monitoring. However, current bulk sequencing methods are unable to unambiguously identify rare pathogenic or drug-resistant cell populations and determine whether mutations co-occur within the same cell. Single-cell sequencing has the potential to provide unique insights on the cellular and genetic composition, drivers, and signatures of cancer at unparalleled sensitivity. Previously we have developed a high-throughput single-cell DNA analysis platform (Tapestri™, Mission Bio, South San Francisco Calif.) that leverages droplet microfluidics and a multiplex-PCR based targeted DNA sequencing approach, and demonstrated the generation of high-resolution maps of clonal architecture from acute myeloid leukemia (AML) tumors.

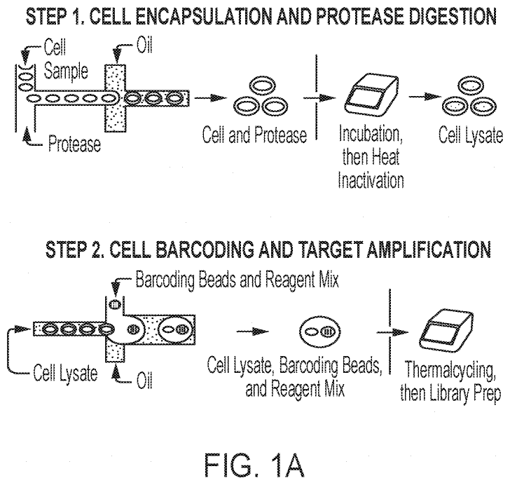

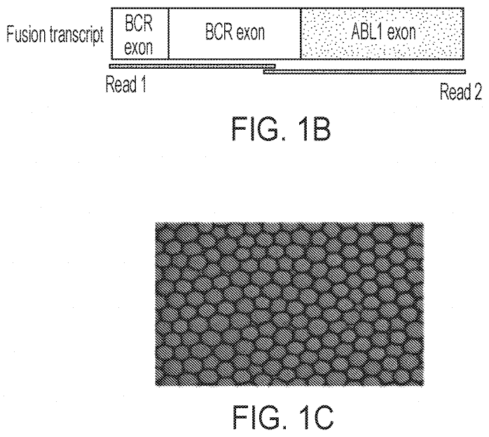

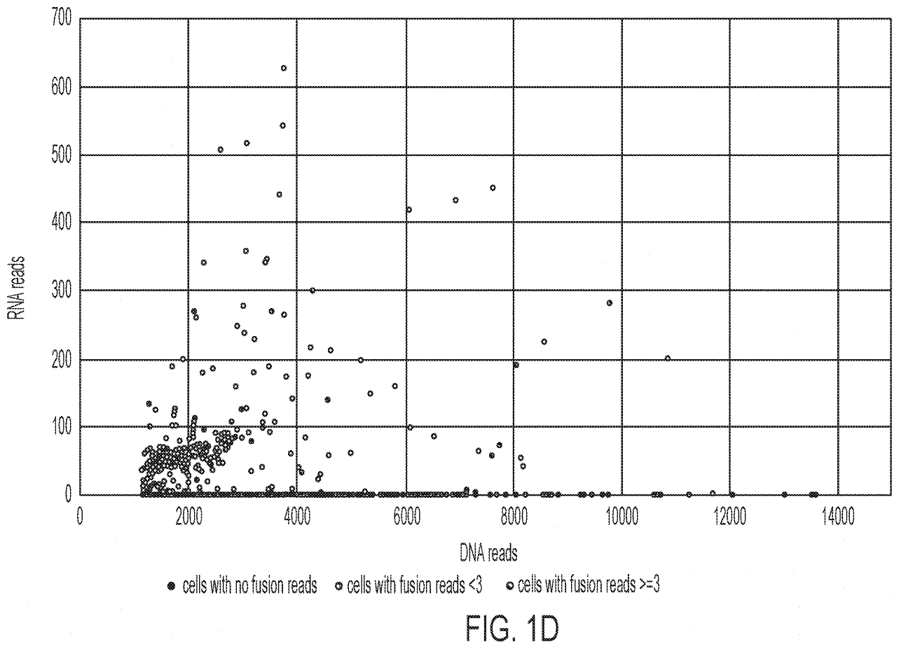

An exemplary embodiment is a system and method for detection of a target nucleic acid mutation or translocation from a single cell, the method including, independent of order presented, the following steps: selecting one or more target nucleic acid sequence, where the target nucleic acid sequence is complementary to a nucleic acid in a cell suspected of having a mutation or translocation; providing a sample having on or more individual single cells; encapsulating one or more individual cell in a reaction mixture comprising a protease; incubating the encapsulated cell with the protease in the drop to produce a cell lysate; providing one or more nucleic acid amplification primer sets, wherein each primer set is complementary to a target nucleic acid and at least one primer of a nucleic acid amplification primer set comprises a barcode sequence; performing a nucleic acid amplification reaction to form an amplification product from the nucleic acid of a single cell, where the amplification product has amplicons of one or more target nucleic acid sequence; providing an affinity reagent that comprises a nucleic acid sequence complementary to the barcode sequence of one of more nucleic acid primer of a primer set, where the affinity reagent comprising said nucleic acid sequence complementary to the barcode sequence is capable of binding to a nucleic acid amplification primer set comprising a barcode sequence; contacting an affinity reagent to the amplification product comprising amplicons of one or more target nucleic acid sequence under conditions sufficient for binding of the affinity reagent to the target nucleic acid to form an affinity reagent bound target nucleic acid; and characterizing a mutation or translocation associated with the target nucleic acid by nucleic acid sequencing (see for example, FIG. 1A).

Some embodiments are directed to methods and systems for the detection of mutations or variants in a cell subclone or in a target nucleic acid or allelic variants thereof. For example, some embodiments are directed to the selection of informative or clinically relevant single cell variants using the methods and systems described herein. Other implementations are directed to the detection of indels (insertion/deletion) and fusion transcripts. One particular fusion transcript of interest is BCR-ABL1, which is used to provide embodiments for the detection of an AML tumor, embodiments for the detection of a leukemia, embodiments for the detection of myeloid leukemia, and embodiments for testing the progression and prognosis as well as treatment for AML tumors, leukemias, myeloid leukemias, and the like. One particular implementation is directed to method for detection of a BCR-ABL1 nucleic acid mutation from a single cell through a method of amplification with selected primers (see FIG. 1B). Some embodiments provided use a method and system as shown in FIG. 1 and as described above, where the target nucleic acid is one that allows detection of a BCR-ABL fusion transcript. Other implementations are directed to a clonal distribution and phylogeny analysis to detect subclones and tumor purity.

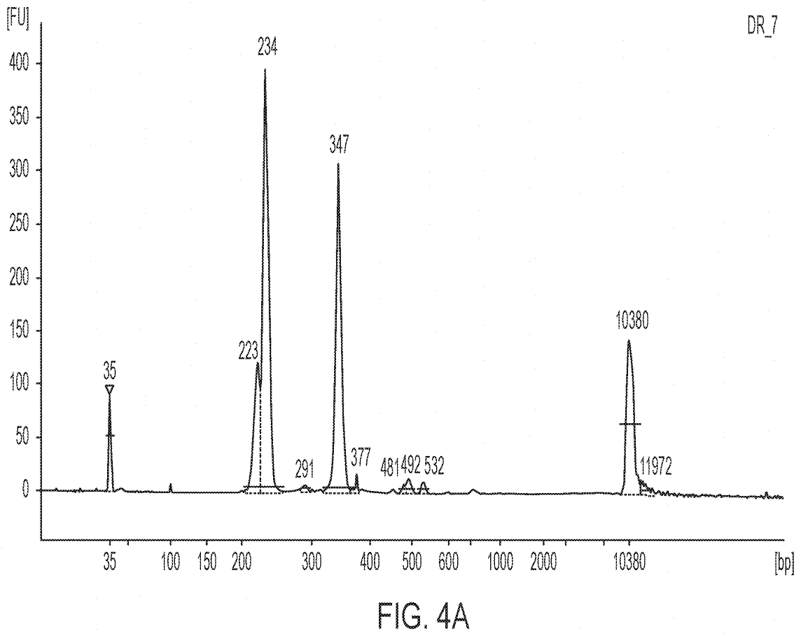

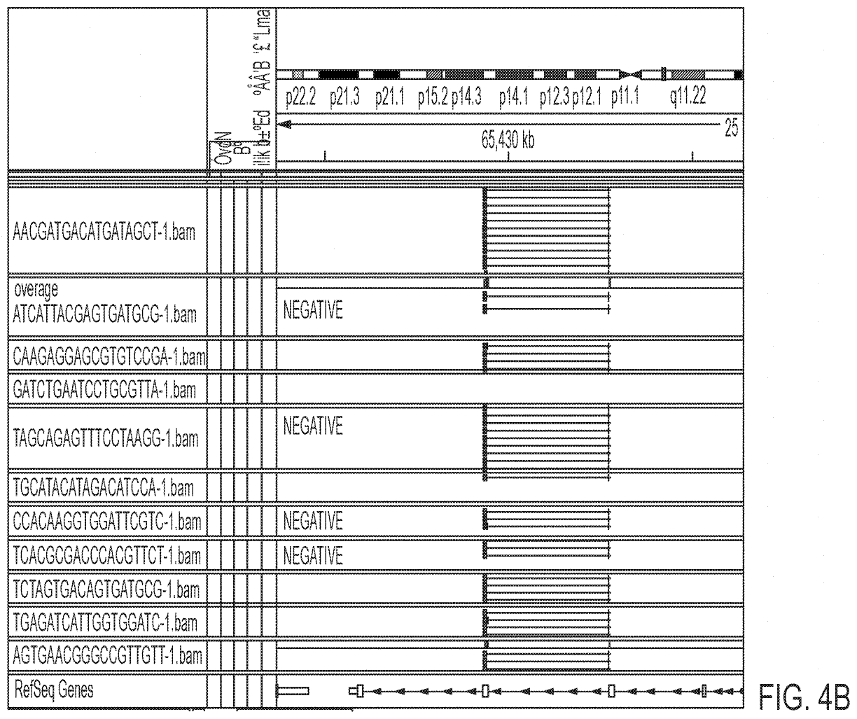

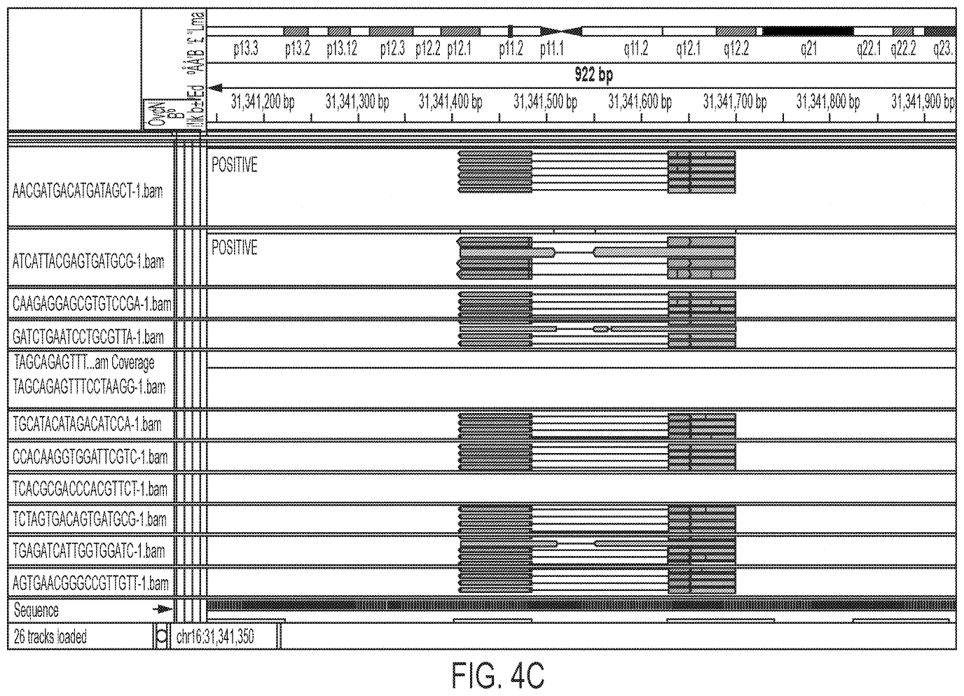

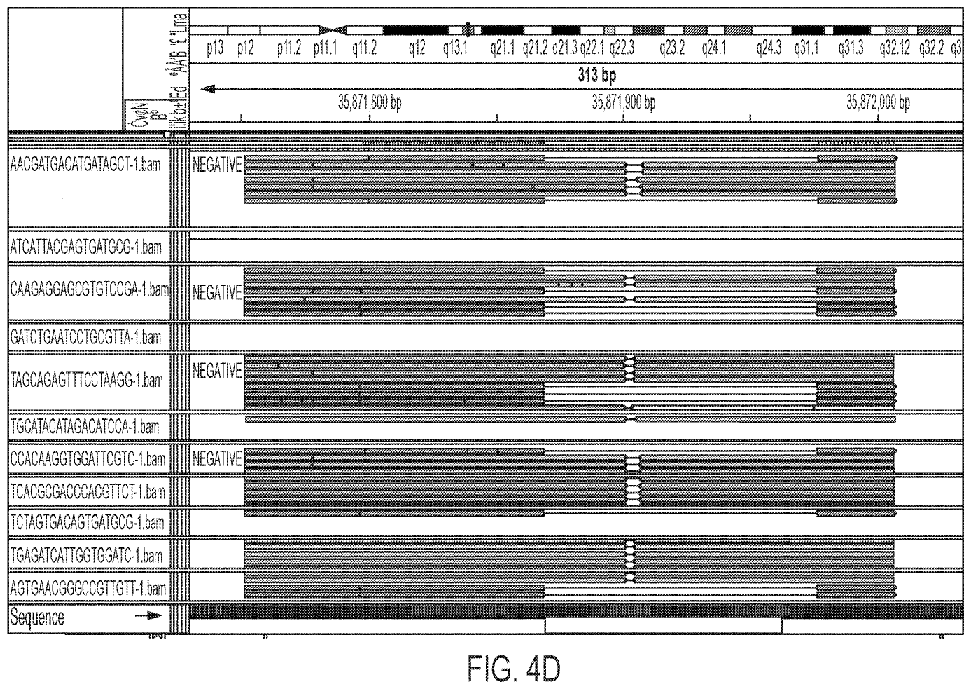

An exemplary embodiment is a system and method for detection of a BCR-ABL gene fusion in a nucleic acid sample from a single cell, the method including, independent of order presented, at least the following steps: selecting one or more target nucleic acid sequence in an individual cell, where the target nucleic acid sequence is suspected of having a BCR-ABL fusion transcript; providing a sample having one or more individual single cell; encapsulating one or more individual cell(s) in a reaction mixture comprising a protease; incubating the encapsulated cell with the protease in the drop to produce a cell lysate; providing a nucleic acid amplification primer set complementary to a target nucleic acid suspected of having a BCR-ABL fusion transcript and where at least one primer of a nucleic acid amplification primer set comprises a barcode identification sequence; performing a nucleic acid amplification reaction to form an amplification product from the nucleic acid of a single cell; and optionally, the following: providing an affinity reagent that comprises a nucleic acid sequence complementary to a barcode sequence of one of more nucleic acid primer, where the affinity reagent comprising said nucleic acid sequence complementary to the barcode sequence is capable of binding to a nucleic acid amplification primer comprising a barcode sequence; contacting an affinity reagent to the amplification product comprising amplicons under conditions sufficient for binding of the affinity reagent to the target nucleic acid to form an affinity reagent bound target nucleic acid; and determining whether the target nucleic acid comprises a BCR-ABL1 fusion transcript (see for example, FIG. 4A).

In another aspect, methods and systems for determining the prognosis of a human patient diagnosed as having or suspected of having a BCR-ABL gene fusion are provided. An exemplary embodiment is a system and method for determining the presence of prognosis of a patient having or suspected of having a BCR-ABL gene fusion in a nucleic acid sample from a single cell. An exemplary embodiment comprises the following steps: selecting one or more target nucleic acid sequence in an individual cell, where the target nucleic acid sequence is suspected of having a BCR-ABL fusion transcript; providing a sample having one or more individual single cell; encapsulating one or more individual cell(s) in a reaction mixture comprising a protease; incubating the encapsulated cell with the protease in the drop to produce a cell lysate; optionally, adding polymerases (e.g. a reverse transcriptase, or active variant thereof), primers and other necessary reaction components needed for performing reverse transcription, and performing a reverse transcription reaction from the nucleic acid of a single cell; providing a nucleic acid amplification primer set complementary to a target nucleic acid suspected of having a BCR-ABL fusion transcript and where at least one primer of a nucleic acid amplification primer set comprises a barcode identification sequence; performing a nucleic acid amplification reaction to form an amplification product from the nucleic acid of a single cell; and determining the prognosis of the patient having or suspected of having a BCR-ABL gene fusion. The prognosis is determined, in part, by nucleic acid sequencing of amplification product(s) in some embodiments. Variations of this approach are used in alternative embodiments to determine the prognosis of a patient suspected of having a myeloproliferative disease. In some embodiments provided herein, amplification primers sets are selected to span and amplify a BCR-ABL splice junction site.

Other aspects of the invention may be described in the follow embodiments:

1. An apparatus or system for performing a method described herein.

2. A composition or reaction mixture for performing a method described herein.

3. A transcriptome library generated according to a method described herein.

5. A genomic and transcriptome library generated according to a method described herein.

6. A kit for performing a method described herein.

7. A cell population selected by the methods described herein.

8. A BCR-ABL gene fusion constructed by the methods described herein.

The following Examples are included for illustration and not limitation.

### Example I

### High Throughput Single Cell Sequencing

In this Example, we provide an embodiment utilizing the Tapestri™ Platform as illustrated in FIG. 1A, on which we have been able to develop a single workflow for the simultaneous detection of DNA and RNA. For the sample preparation, fresh K-562 cells (ATCC) were prepared in the Mission Bio cell buffer. Viability and cell count were determined on the Countless II™ automated cell counter (Thermo Fisher Scientific). 2490 cells/uL were loaded onto a Tapestri™ instrument. Encapsulation of these single cells was performed using Mission Bio reagents. After the cell lysis and protease treatment, cell barcoding and target amplification were performed using primers targeting BCR-ABL1 spiked into a Tapestri™ Single-Cell DNA Acute Myeloid Leukemia Panel (50 amplicons) and the SuperScript™ IV One-Step RT-PCR System. The BCR-ABL1 reverse primer was at 4× the concentration of the DNA reverse primers while the BCR-ABL1 forward primer was at 1× the concentration of the DNA forward primers. RT was performed for 10 minutes at 50 C then the PCR for the targeted amplification was performed. The BCR-ABL1 primers targeted the b3a2 fusion transcript found in K-562 to produce a 237 bp amplicon. Library preparation was completed, resulting in targeted amplicons from both DNA and RNA with sequencing adaptors and dual indexes. As performed in this Example, from cell prep to sequencing-ready libraries targeting both DNA and RNA.

FIG. 1B schematically illustrates an embodiment for the detection of BCR-ABL1 and shows a fusion transcript with paired reads (read 1 and read 2) from sequencing. The primers were designed to cover the BCR-ABL1 b3a2 fusion transcript found in K-562. Read one covers two exons in BCR verifying the read is from RNA. Read 2 covers the splice junction. In this example, the BCR-ABL1 primers with K-562 cells would produce an amplicon of 237 bp from the fusion transcript.

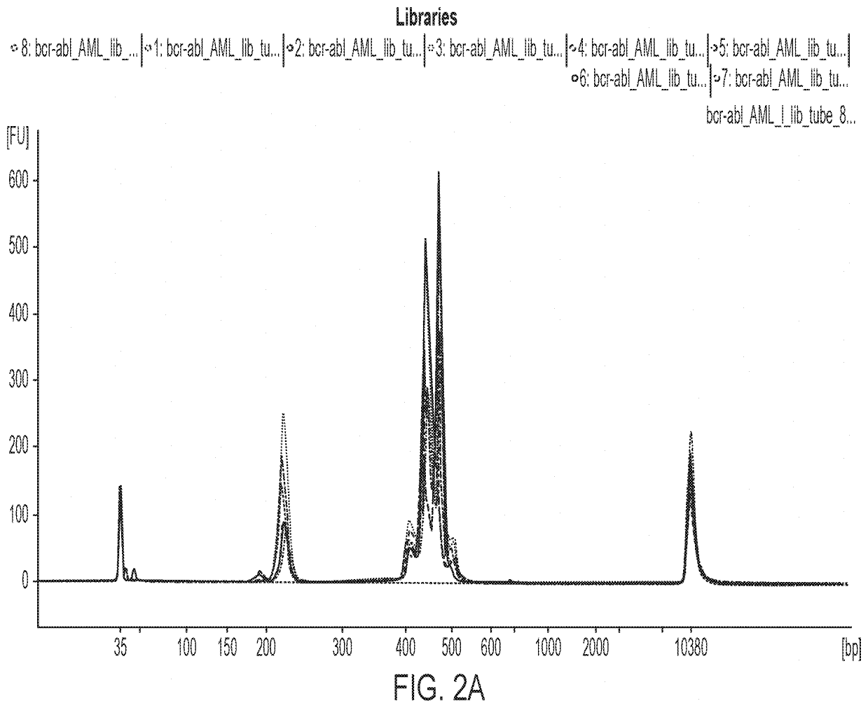

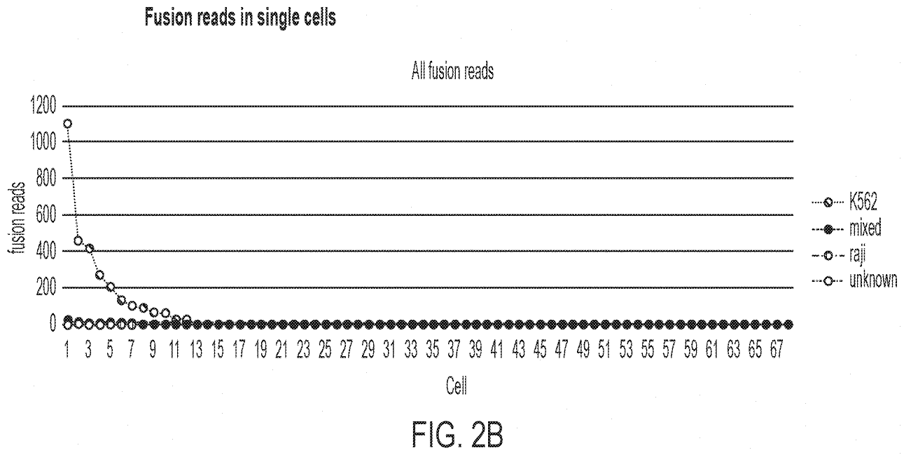

Table 1 shows the primers used in the amplification producing the fusion transcript illustration shown in FIG. 2B.

TABLE 1
BCR-ABL1 amplicon

| Fwd Primer | Rev Primer | Amplicon |
|---|---|---|
| ACTCCAGACT | TTGGGGTCATTTT | ACTCCAGACTGTCCACAGCATTCCGCTGACCATCAATAAG |
| GTCCACAGCA | CACTGGGTCCAG | GAAGATGATGAGTCTCCGGGGCTCTATGGGTTTCTGAATG |
| (SEQ ID NO:) | CGAGAAGGT | TCATCGTCCACTCAGCCACTGGATTTAAGCAGAGTTCAAA |
| | (SEQ ID NO:) | AGCCCTTCAGCGGCCAGTAGCATCTGACTTTGAGCCTCAG |
| | | GGTCTGAGTGAAGCCGCTCGTTGGAACTCCAAGGAAAAC |
| | | CTTCTCGCTGGACCCAGTGAAAATGACCCCAA (SEQ ID |
| | | NO:) |

The droplets formed on Tapestri after the targeted RT-PCR cycling is shown in FIG. 1C demonstrating that single cell lysates and reaction components remain partitioned throughout the reaction.

FIG. 1D shows the codetection of sequencing reads from both the 50 plex AML DNA panel and the RNA amplicon detecting the b3a2 fusion transcript from 1423 cells. 37.5% of the cells genotyped as a K-562 cell had 3 or more fusion reads sequenced per cell.

In FIG. 1E, we show similar DNA panel performance independent of fusion detection.

### Example II

### Method and System for Fusion in DNA and RNA

This Example shows results and a workflow using Tapestri™ for the analysis of genetic and allelic variations from both DNA and RNA. K-562 cells (ATCC) and Raji cells (ATCC) were used to demonstrate fusions from RNA can be detected concordantly with insertions/deletions and SNVs in DNA. 2690 cells/uL of a 50:50 mix of K-562 and Raji cells were prepared in the Mission Bio cell buffer and loaded on the Tapestri™ instrument. After encapsulation, lysis, and protease treatment of these single cells using Mission Bio reagents, targeted amplification was performed. The Tapestri™ Single-Cell DNA Acute Myeloid Leukemia Panel (50 amplicons) was supplemented with BCR-ABL1 primers for the RT-PCR. The BCR-ABL1 reverse primer was at 2× the concentration of the DNA reverse primers while the BCR-ABL1 forward primer was at 1× the concentration of the DNA forward primers. SuperScript™ IV One-Step RT-PCR System was used for the RT-PCR. RT was performed for 10 minutes at 45 C followed by targeted PCR. The BCR-ABL1 primers targeted the b3a2 fusion transcript found in K-562 to produce a 237 bp amplicon. Library preparation was performed, resulting in a single library with both DNA and RNA from each tube output from the Tapestri™ instrument, as shown in FIG. 2A.

Table 2 shows BCR and ABL1 paired reads that were aligned to the fusion amplicon. There were no false positives when requiring 3 or more fusion reads sequenced per cell to confirm the presence of the fusion in the cell. The graphical version of Table 2 is seen in FIG. 2B where 3 or more fusion reads per single cell were sequenced only in K-562 cells or mixed cells where the cells mixed are K-562 and Raji cells.

TABLE 2
Fusion reads associated with cells

| | | Cells with | Cells with | Cells with |
|---|---|---|---|---|
| | Total | fusion | fusion | fusion |
| Cell Types | Cells | reads | reads >= 2 | reads >= 3 |
| K-562 | 255 | 31 | 18 | 14 |
| Mixed | 186 | 68 | 34 | 19 |
| Raji | 146 | 7 | 0 | 0 |
| Unknown | 20 | 4 | 1 | 0 |

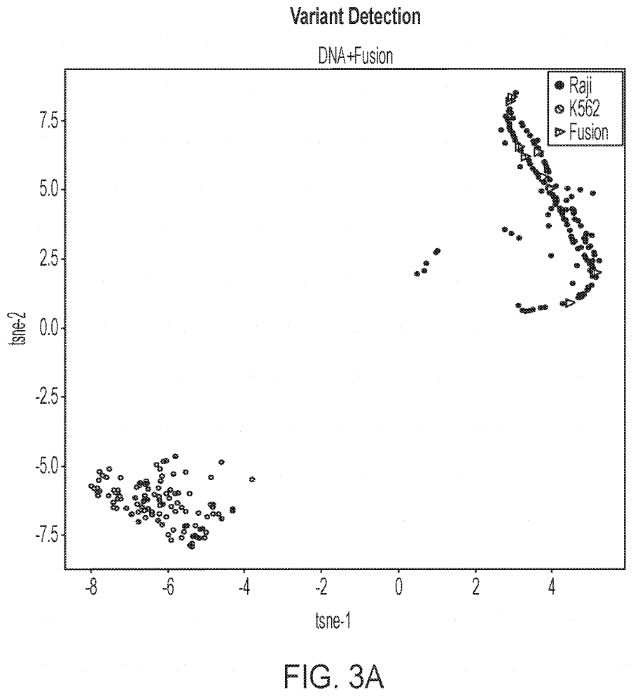

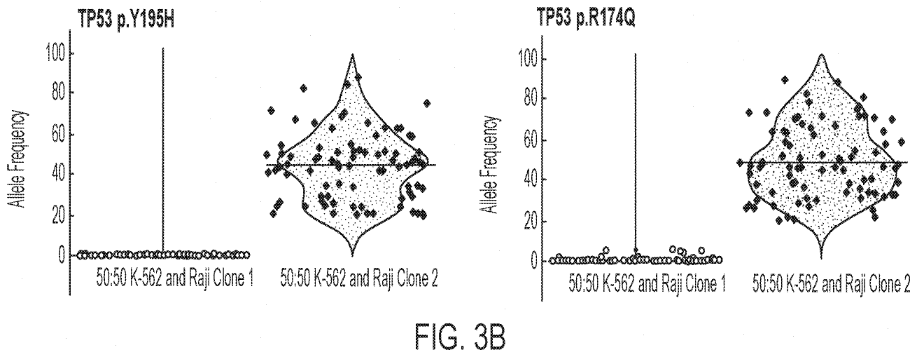

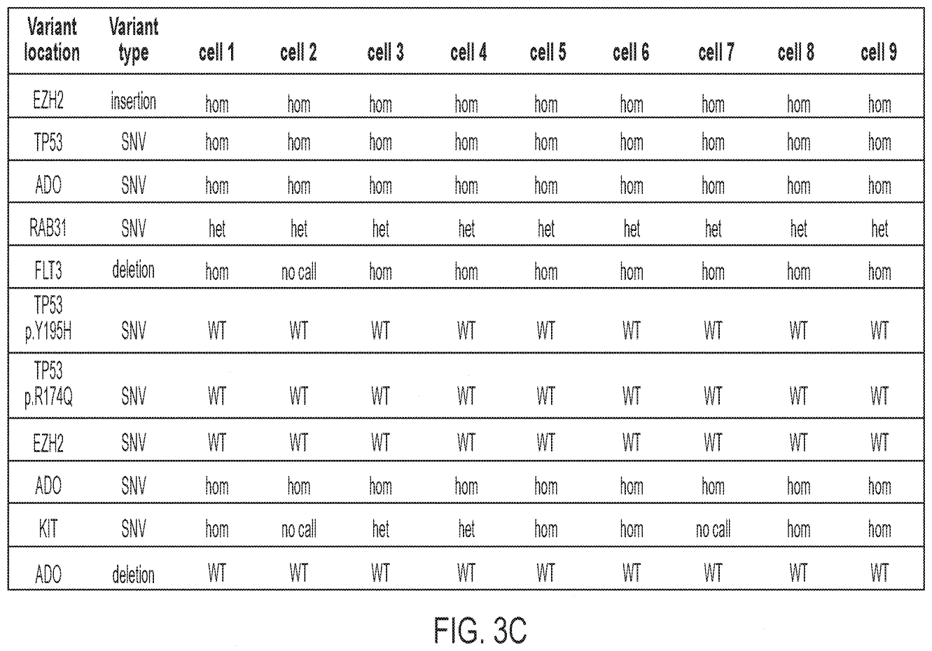

The 50:50 mix of K-562 and Raji cells cluster based on cell type in a t-SNE plot in FIG. 3A using 11 SNVs and indels. Cells genotyped as K-562 and Raji are shown in red and blue, respectively. 3 or more BCR-ABL1 fusion reads were found only in the K-562 cells, shown in green. The expected DNA variants for the K-562 and Raji cells were also successfully found. FIG. 3B shows the K-562 and Raji cells separate into clones where Clone 1 is the expected allelic frequency for K-562 and Clone 2 is the expected allelic frequency for Raji. All 11 SNVs are shown in FIG. 3C where the expected variant calls for each cell with 3 or more BCR-ABL1 fusion reads were detected. Positions on the genome for allelic drop out analysis were abbreviated as ADO.

### Example III

### DR005

This Example shows results and a workflow using Tapestri™ for the analysis of gene expression from RNA using RT-PCR. KG-1 cells were used with a 58 plex gene expression panel to gene expression from multiple targets can be detected using the Tapestri™ instrument to partition the cells and perform targeted amplification. 3000 cells/uL KG1 cells in Mission Bio cell buffer were input on the Tapestri™ instrument. Encapsulation and cell lysis of these single cells were performed. SuperScript™ IV One-Step RT-PCR System was used for the RT-PCR with the 58 plex gene expression panel primers. RT was performed for 10 minutes at 50 C followed by targeted PCR. Library preparation was performed, resulting in a single library with both DNA and RNA from each tube output from the Tapestri™ instrument, as shown in FIG. 4A.

Table 3 shows the genes targeted from the 58 plex gene expression panel.

TABLE 3
58 plex gene expression panel

| | | | | | | | | |
|---|---|---|---|---|---|---|---|---|
| CCL22 | CDK1 | CD27 | TBX21 | FASLG | MKI67 | IDO1 | CD69 | PTGS2 |
| TNFSF4 | NFKBIA | CXCL10 | CD274 | CA4 | BCL2 | CD40LG | ITGB2 | SIT1 |
| PRF1 | ITGAM | DDX58 | IL10 | STAT3 | CCL2 | HLA-C | SAMHD1 | |
| IL7R | MTOR | AKT1 | GUSB | IL12A | CD80 | HGF | KLRD1 | |
| LAG3 | ZAP70 | LAMP3 | IL4 | CD8A | TNFRSF4 | RORC | TLR3 | |
| HLA-A | IFNG | CD3E | CD28 | FOXO1 | HIF1A | PTEN | IL6 | |
| BRCA1 | CD86 | CXCL1 | TNFSF9 | CCR5 | VCAM1 | STAT1 | TNFRSF14 | |

Sequencing reads were paired and aligned to hg19 using STAR on DNA nexus. FIG. 4B is an image from the Integrative Genomics Viewer (Broad Institute) of the reads aligned to the GUSB gene separated based on cell barcode where each cell barcode is unique to a single cell. The reads align to the exons, shown in blue, with no reads crossing into the intron as expected from reads originating from RNA. FIG. 4C shows the ITGAM gene with the same single cells on the Integrative Genomics Viewer. The reads align to the exons, shown in blue, with few reads crossing into the intron. The reads crossing into the intron could be from DNA while the reads aligning only in the exons are from RNA. FIG. 4D shows the NFKBIA gene with the same single cells on the Integrative Genomics Viewer. There is a mix of reads containing both exonic and intronic reads. The reads aligning only in the exons are from RNA while those with both exon and intron regions could be from DNA.

### Example IV

### DR010

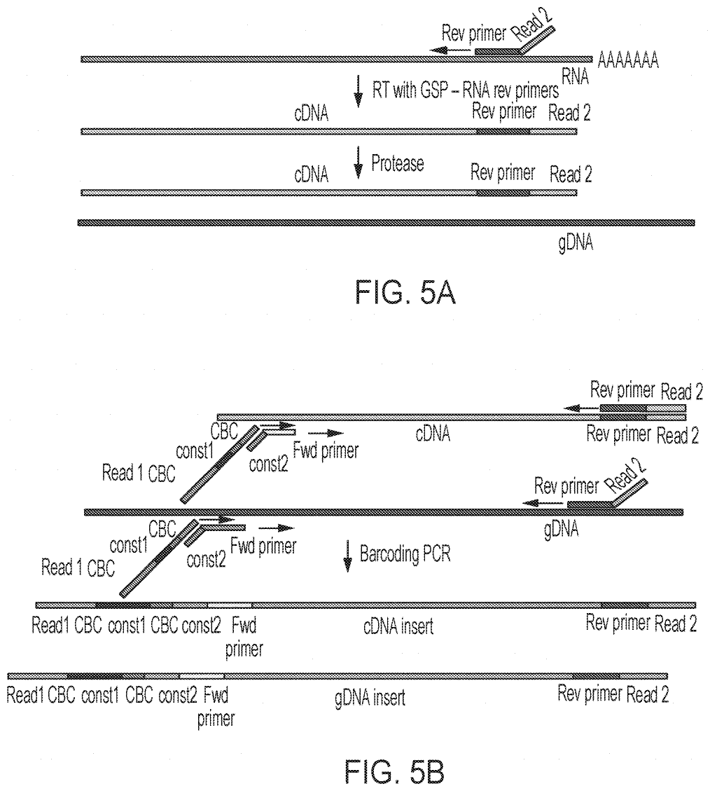

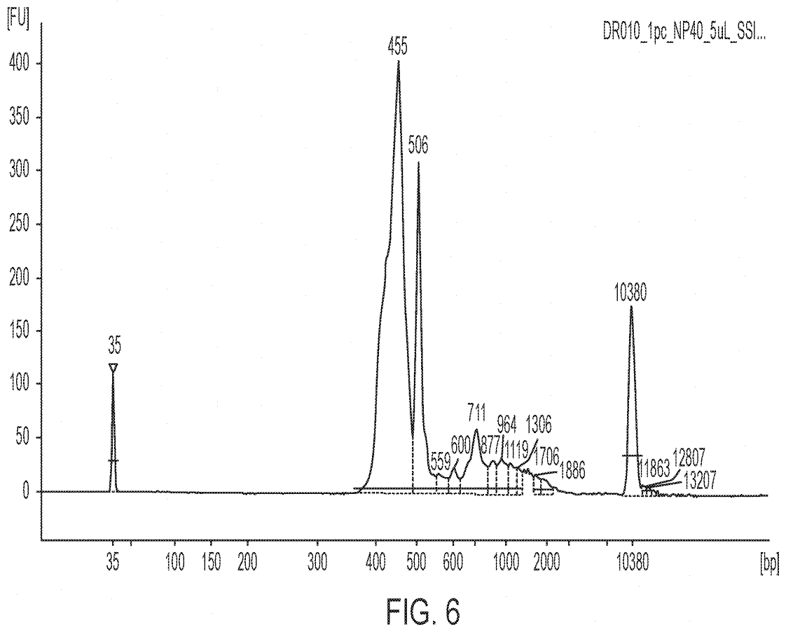

This Example shows results and a workflow using Tapestri™ for the analysis of genetic and allelic variations from both DNA and RNA using reverse transcription followed by targeted PCR to detect multiple RNA targets. This example demonstrates a second workflow where the reverse transcription is performed in a different microdroplet than the targeted PCR and it also demonstrates targeting multiple fusion transcripts in a single reaction concurrently with DNA variants. A mix of K-562, TOM-1, and Raji cells were prepared in the Mission Bio cell buffer and loaded on the Tapestri™ instrument. In the first droplet, which encapsulated the single cells, cell lysis, reverse transcription, and protease treatment was performed. Reverse transcription was performed with the SuperScript™ IV First-Strand Synthesis System and a single primer targeting three BCR-ABL1 fusions transcripts. These droplets were then input into the Tapestri™ Instrument for droplet merger for targeted PCR. The Tapestri™ Single-Cell DNA Acute Myeloid Leukemia Panel v2 (128 amplicons) was spiked in with with BCR-ABL1 forward primers for the targeted PCR. A schematic is shown in FIGS. 5A-5B where FIG. 5A shows the first droplet reaction and FIG. 5B shows the second droplet reaction. Library preparation was performed, resulting in a single library with both DNA and RNA sets of 4 tubes from the Tapestri™ instrument, as shown in FIG. 6.

Table 4 shows the targeted fusions and amplicon sizes with the primers used. A single primer was used for reverse transcription and two primers were used for the forward primers. K-562 has the p210 b3a2 fusion transcript and TOM-1 has the p190 fusion transcript. The b2a2 fusion transcript was not present in this example.

| Fusion | Amplicon size |
|---|---|
| p190 | 270 |
| p210 b2a2 | 241 |
| p210 b3a2 | 316 |

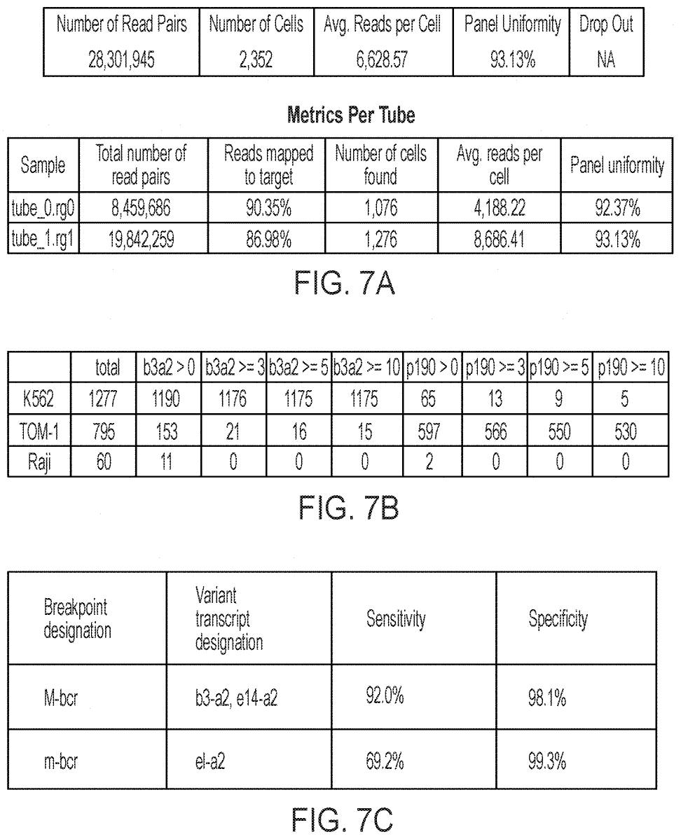

4 out of the 8 tubes from Tapestri™ were sequenced with 2352 single cells identified, seen in FIG. 7A. 3 single nucleotide variations were used to differentiate the 3 cell lines resulting in 2181 genotyped cells (FIG. 7B). FIG. 7C shows the sensitivity and specificity calculations for these genotyped cells. With 5 fusion reads required for a positive call, the sensitivity for p210 b3-a2 detection was 92.0% and for p190 e1-a2 detection was 69.2%. Both had specificities of greater than 98%. Raji had no fusions called.

### Example V

### DR014

This Example shows results and a workflow using Tapestri™ for the analysis of genetic and allelic variations from both DNA and RNA using reverse transcription followed by targeted PCR to detect multiple RNA targets. This example demonstrates the detection of 3 distinct fusion transcripts in a single reaction concurrently with DNA variants. A mix of K-562, TOM-1, KCL-22 and KG-1 cells were prepared as described in Example 4. K-562 contains the p210 b3a2 fusion transcript. TOM-1 contains the p190 fusion transcript. KCL-22 contains the p210 b2a2 fusion transcript and KG-1 has no fusion transcripts. The protocol for Tapestri™ was as described in Example 4 but with twice the concentration of forward primer for the BCR-ABL1 fusion transcripts.

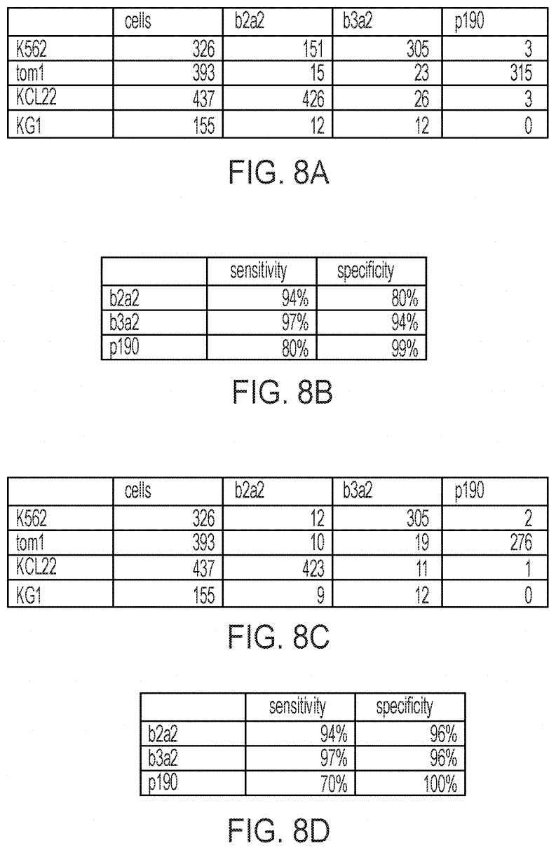

4 out of the 8 tubes from Tapestri™ were sequenced with 1898 single cells identified with 1311 cells genotyped with 3 single nucleotide variants. FIG. 8A shows the fusion called in cells genotyped with more than 5 fusion reads sequenced per cell. The corresponding sensitivity and specificity calculations for these genotyped cells are in FIG. 8B. With 20 fusion reads required for a positive call (FIG. 8C), the sensitivity for p210 b3-a2 detection was 97.0%, for p210 b2a2 was 94% and for p190 e1-a2 detection was 70%. All specificities were 96% or above.

### Example VI

### DR015

This Example shows results and a workflow using Tapestri™ for the analysis of genetic and allelic variations from both DNA and RNA using reverse transcription followed by targeted PCR to detect multiple RNA targets with reverse transcription primers with differing annealing temperatures. This example demonstrates the use of the workflow and chemistry the reverse transcription is performed in a different microdroplet than the targeted PCR to detect gene expression concurrently with variants from the DNA from those same single cells. This example also demonstrates the ability to separate the DNA and RNA libraries from the same single cell and pair them based on their cell barcodes. Additionally, this example demonstrates the flexibility of annealing temperature of the reverse transcription primers.

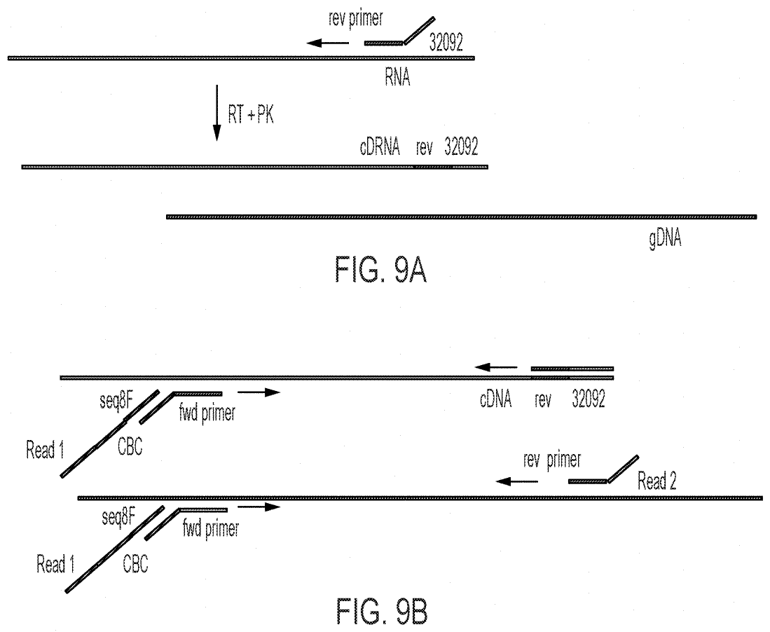

A mix of KG-1, Jurkat, and Y79 cells were prepared in the Mission Bio cell buffer and loaded on the Tapestri™ instrument. In the first droplet, which encapsulated the single cells, cell lysis, reverse transcription, and protease treatment was performed. In one reaction reverse transcription was performed with the SuperScript™ IV First-Strand Synthesis System and the reverse transcription primers from a 110 plex gene expression panel with an annealing temperature of 63 C. In a second reaction, reverse transcription was performed with the SuperScript™ IV First-Strand Synthesis System and the reverse transcription primers from a 110 plex gene expression panel with an annealing temperature of 45 C. These droplets were then input into separate Tapestri™ Instruments for droplet merger for targeted PCR. The 88 plex DNA panel was combined with the forward primers for the 110 plex gene expression panel. A schematic is shown in FIGS. 9A-9B where FIG. 9A shows the first droplet reaction and FIG. 9B shows the second droplet reaction. Library preparation was performed, resulting in separated paired libraries, one with targeted DNA amplicons and the other with targeted RNA amplicons from 4 tubes from the Tapestri™ instrument. The gene expression targets are shown in Table 5.

TABLE 5

| | | | | | |
|---|---|---|---|---|---|
| CD8A | CD33 | GYPA | IL2RA | PDCD1 | TOX |
| CCR7 | CD276 | HIF1A | IL12A | PDCD1LG2 | VEGFA |
| CCR5 | CCR2 | HLA-C | IL7 | BRCA1 | TLR9 |
| CD40LG | CD80 | HAVCR2 | IL17A | BTLA | TLR3 |
| CD1C | CD274 | FOXO1 | KLF4 | PTGS2 | TRIM29 |
| CD5 | CD101 | GZMB | IRF4 | PVR | ZC3H12C |
| CCR4 | CLEC4C | HLA-B | ITGB2 | PTEN | VCAM1 |
| CD19 | CXCL2 | HLA-A | KLRK1 | PRF1 | CCL3 |
| CD8B | CXCL1 | HLA-DRA | KLRG1 | SAMHD1 | XCL2 |
| CD7 | CX3CL1 | ICOS | LAMP3 | SLAMF7 | CCL2 |
| CD28 | CX3CR1 | ICAM1 | BCL2 | SLC2A1 | |
| CD200R1 | CXCL10 | IFNG | LAG3 | SLC16A1 | |
| CD86 | CTLA4 | IL10 | MKI67 | SLAMF6 | |
| CD48 | CXCL11 | IL15RA | MPO | SMAD7 | |
| CD244 | CXCR5 | IL4 | NKG7 | STAT4 | |
| CD2 | ENTPD1 | IL1B | NFKBIA | TNF | |
| CDK1 | FCER1A | IL5 | NCR1 | TBP | |
| CD14 | FCGR3A | IL13 | NOS2 | TNFSF4 | |
| CD160 | FASLG | IKZF2 | NT5E | TIGIT | |
| CCL8 | AREG | IL6 | BRCA2 | TNFRSF9 | |

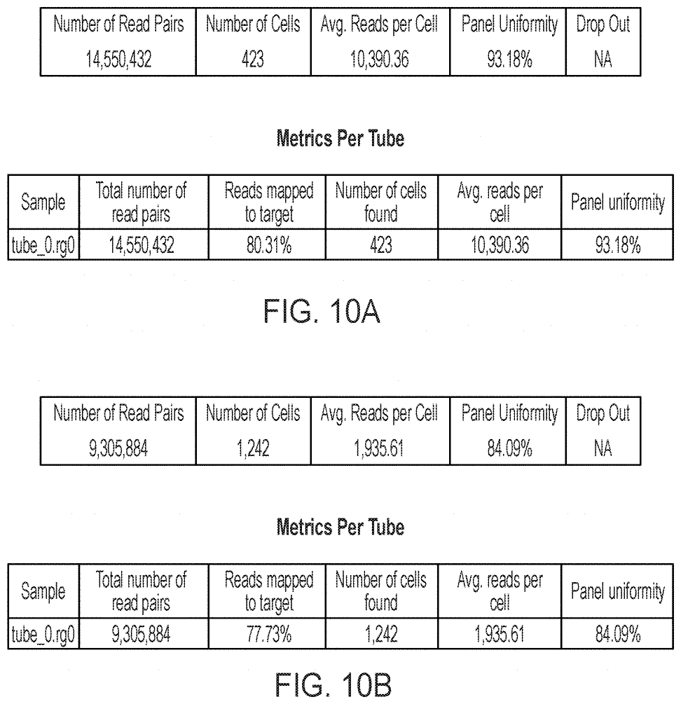

The DNA panel performance, shown in FIGS. 10A-10B, from both reactions shows that a high percentage of reads align to the expected genomic targets with good amplicon uniformity. FIG. 10A shows the results from the reverse transcription primers with annealing temperatures of 63 C while FIG. 10B shows the results from the reverse transcription primers with lower annealing temperatures of 45 C.

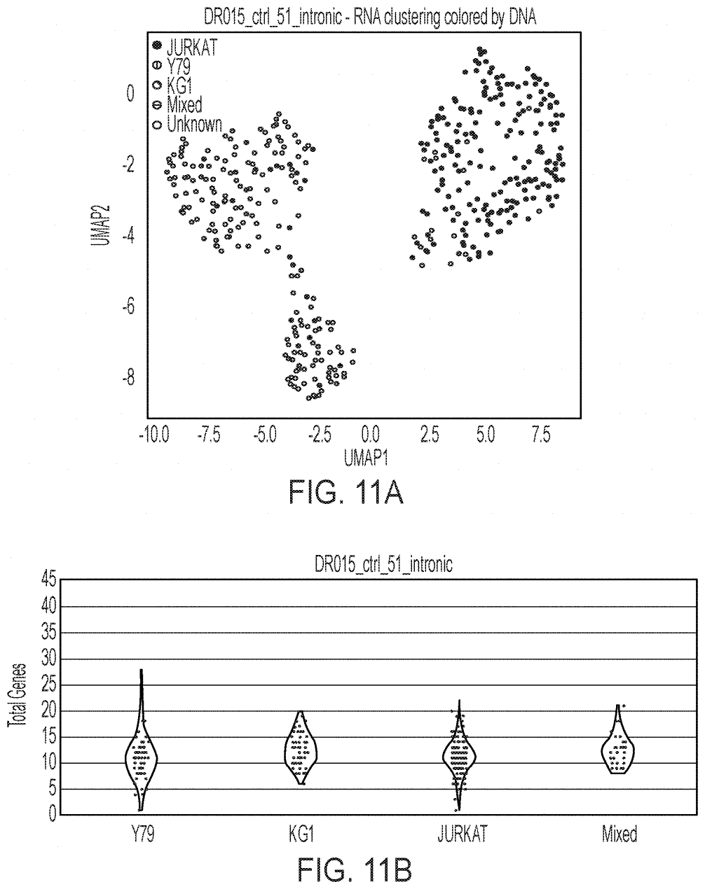

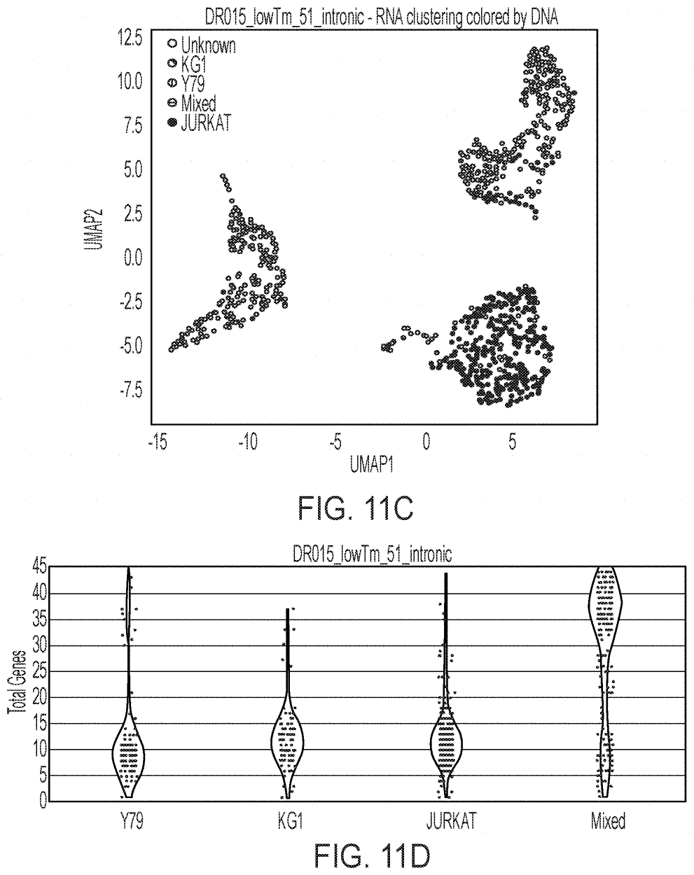

Gene expression reads were detected with both reactions. The number of genes per cell detected from cells types identified by their single nucleotide variants from the DNA library sequencing results were calculated. FIG. 11A shows the results from the reverse transcription primers with an annealing temperature of 63 C while FIG. 11C shows the results from the primers with the 45 C annealing temperature.

FIGS. 11B and 11D show the agreement between cell type data from DNA reads and RNA reads. The cells were clustered with umap based on the RNA library sequencing results then each cell colored based on the cell type identified by the DNA library single nucleotide variant data.

### Example VII

### DR012

This Example shows results and a workflow using Tapestri™ for the analysis of genetic and allelic variations from both DNA and RNA using reverse transcription followed by targeted PCR to detect multiple RNA targets. This example demonstrates the use of the workflow and chemistry the reverse transcription is performed in a different microdroplet than the targeted PCR to detect gene expression concurrently with variants from the DNA from those same single cells. This example also demonstrates the ability to separate the DNA and RNA libraries from the same single cell and pair them based on their cell barcodes. A mix of K-562 and Y79 cells were prepared in the Mission Bio cell buffer and loaded on the Tapestri™ instrument. In the first droplet, which encapsulated the single cells, cell lysis, reverse transcription, and with and without protease treatment was performed. Reverse transcription was performed with the SuperScript™ IV First-Strand Synthesis System and the reverse transcription primers from a 915 plex gene expression panel. These droplets were then input into separate Tapestri™ Instruments for droplet merger for targeted PCR. The 88 plex DNA panel was combined with the forward primers for the 915 plex gene expression panel. This example follows the schematic is shown in FIG. 9 where FIG. 9A shows the first droplet reaction and FIG. 9B shows the second droplet reaction. Library preparation was performed, resulting in separated paired libraries, one with targeted DNA amplicons and the other with targeted RNA amplicons from 4 tubes from the Tapestri™ instrument.

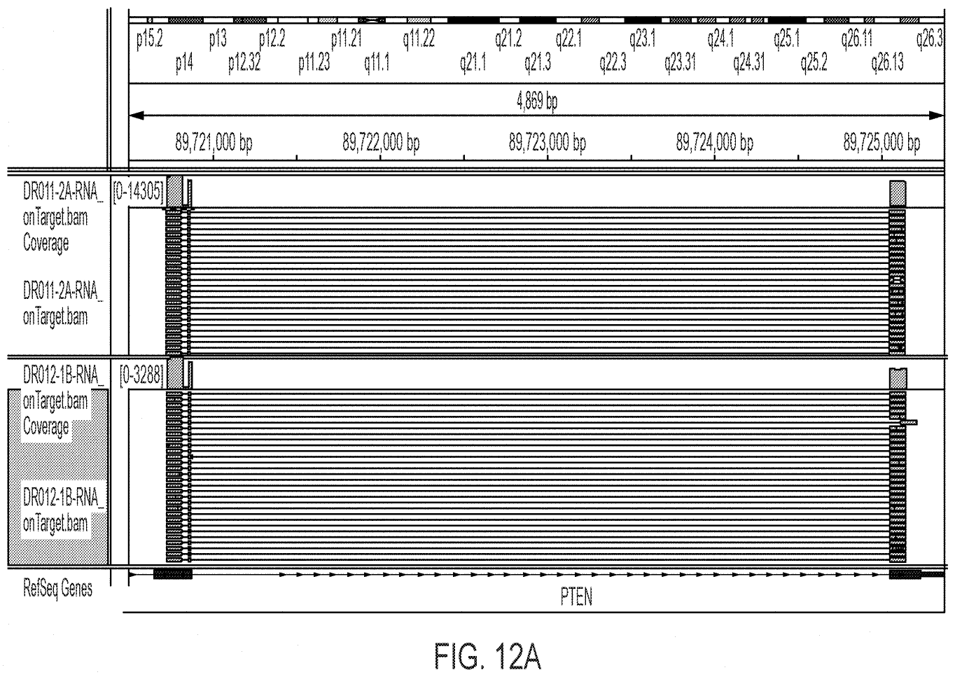

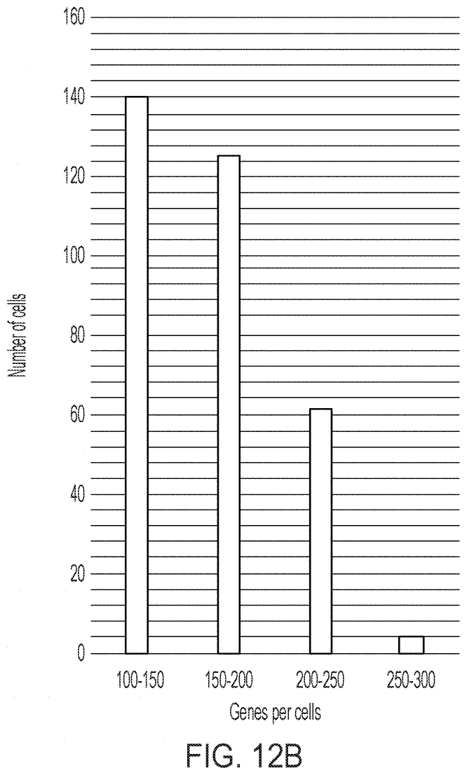

The RNA libraries were produced from RNA reads, shown in FIG. 12. FIG. 12A is an image from the Integrative Genomics Viewer (Broad Institute) of the reads aligned to the PTEN gene where the top panel is the library from the sample where protease was not present and the bottom panel was where protease was used. The sequencing reads align to the exons for both reactions and do not cross into the introns demonstrating they are from RNA molecules. FIG. 12B shows the number of genes detected in each single cell for the sample where protease was present.

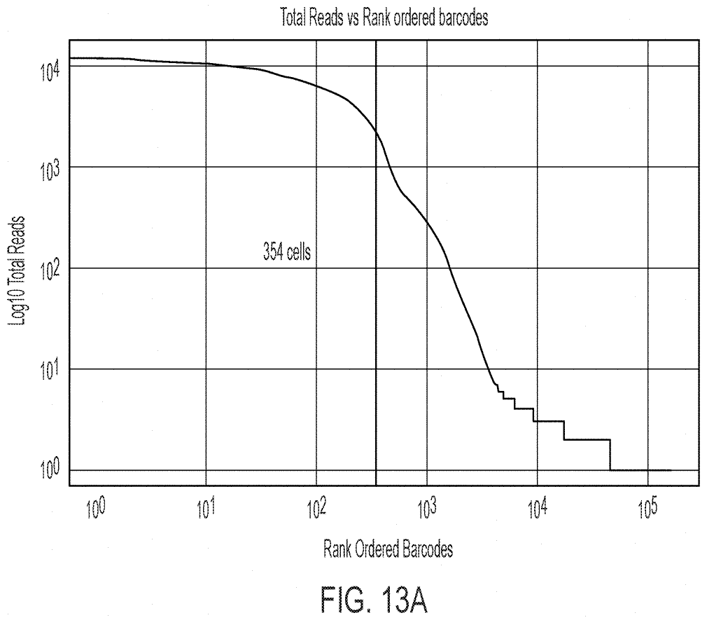

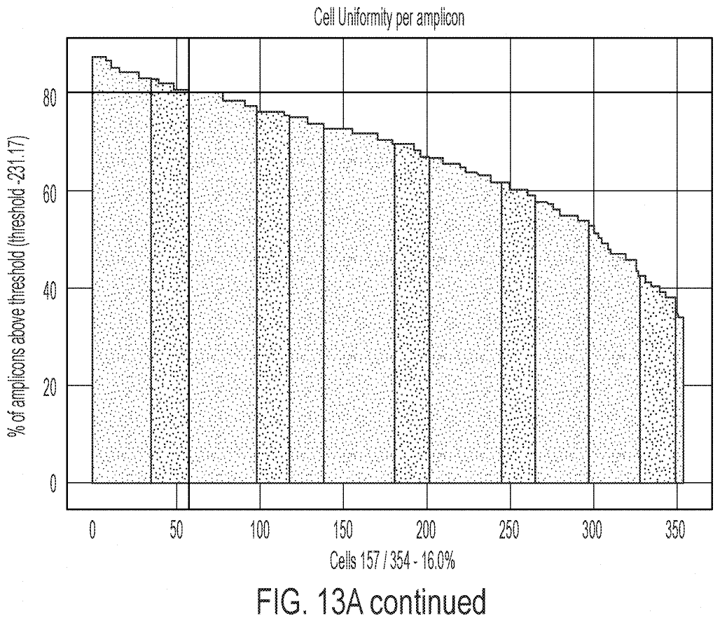

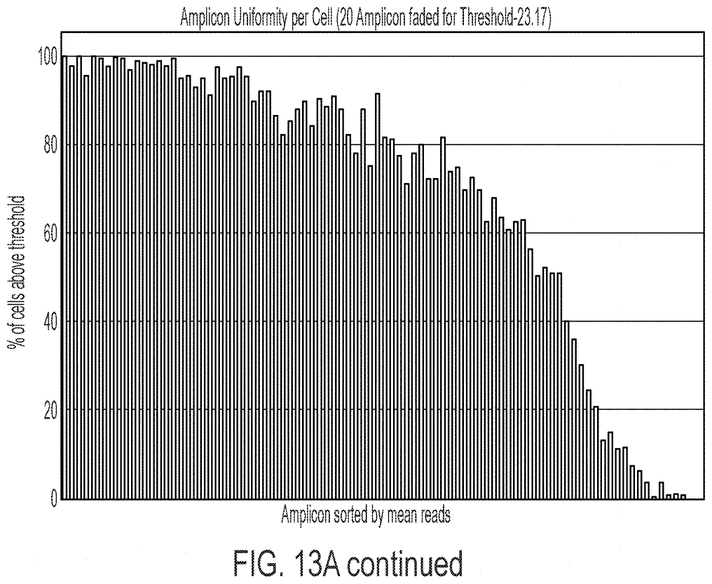

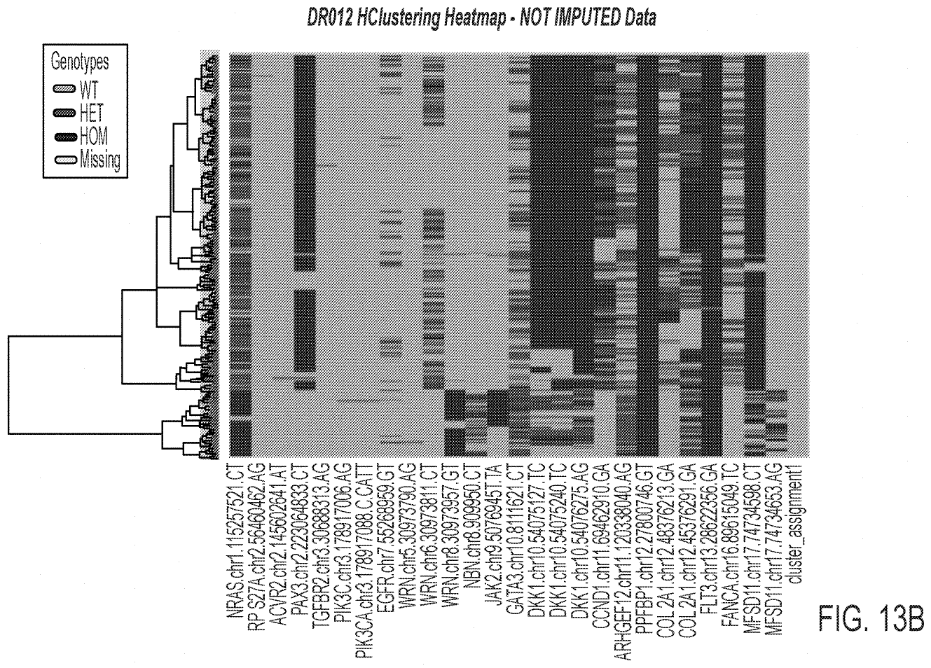

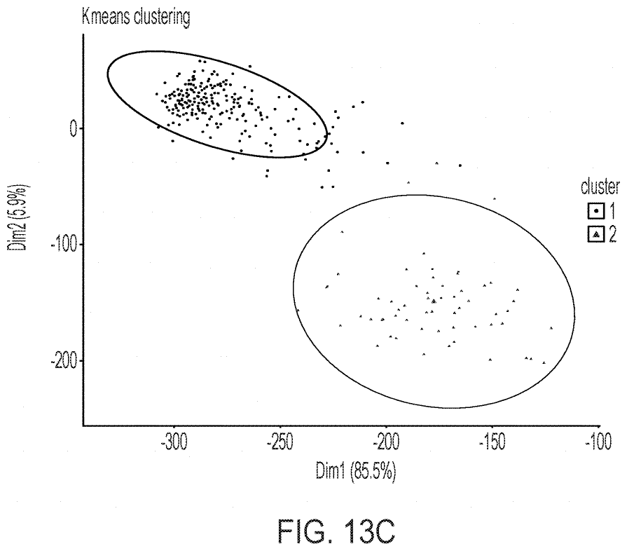

FIG. 13 show the performance of the DNA library from the same 354 cells shown in FIG. 12B. FIG. 13A shows the cell finder threshold, the uniformity of cells, and the uniformity of the amplicons. FIG. 13B shows that single nucleotide variants can be detected in the same DNA libraries where the cells also had RNA libraries. 13 C shows these same cells can be clustered based on their single nucleotide variants.

High Throughput Sequencing with On-Bead Primer Panels

As stated, a barcode sequence can additionally be incorporated into microfluidic beads to decorate the bead with identical sequence tags. Such tagged beads can be inserted into microfluidic droplets and via droplet PCR amplification, tag each target amplicon with the unique bead barcode. Such barcodes can be used to identify specific droplets upon a population of amplicons originated from. This scheme can be utilized when combining a microfluidic droplet containing single individual cell with another microfluidic droplet containing a tagged bead.

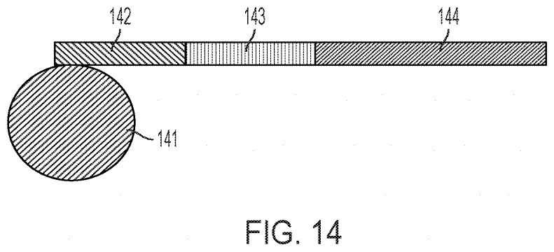

In one embodiment, the disclosure provides method, system and apparatus for custom panels including beads with one or more primers attached to the outside of the bead. FIG. 14 is a schematic illustration of an exemplary embodiment of a bead with an externally-linked primer. In FIG. 14, bead 1410 schematically represents a bead. Bead 1410 may comprise conventional beads having an external surface. The bead chemistry may be varied according to the desired application. Conventional bead include, for example, acrylamide beads with crosslinked acrydite oligonucleotides (“oligos”). The oligos can define synthetic oligos. The oligos can be bound to the beads during the bead preparation or in commercial beads. The size of beads can range from 60-80 um, but other sizes can be used, for example, beads may be in a range of about 1-100 um. Other examples of beads include the use of commercial beads that after chemical treatment can bind oligos with an amino base.

Referring again to FIG. 14, bead 1410 is linked to portion 1420 of primer 1400. The attachment may comprise chemical attachment and/or bonding. For example, Oligos containing the barcodes can be added to the beads by, for example, PCR extension with sequences complementary to the oligo bound to the bead. In other cases the barcodes can be added by ligation to the oligo of the beads.

Portion 1420 of FIG. 14 may comprise a paired sequencing read (e.g., read 1 and read 2, FIG. 1). Portion 1430 of FIG. 14 may comprise a barcode (BC). The barcode can be a generic barcode configured for detection and read.

Portion 1430 of primer 1430 may comprise a common sequence. The commons sequence may be configured for a specific application.

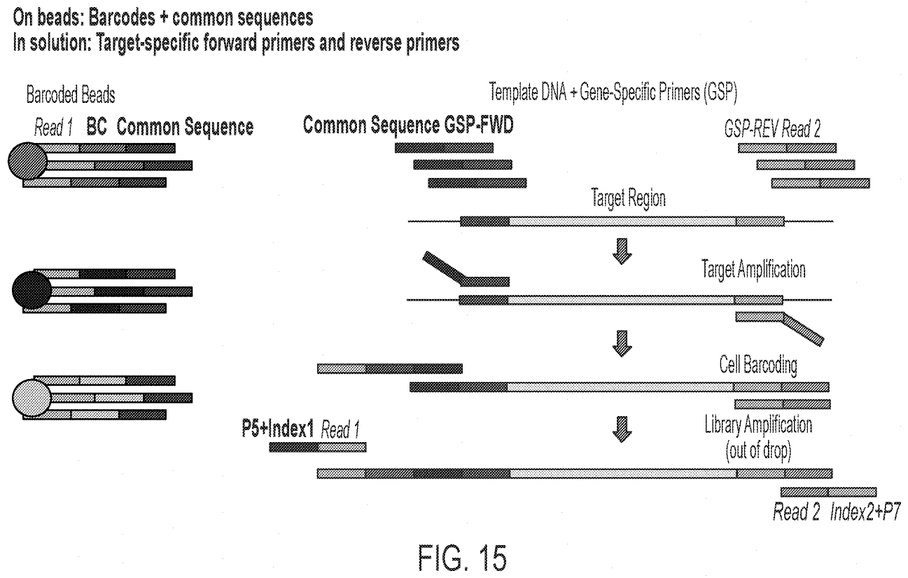

FIG. 15 is an illustration of an exemplary application of an externally-linked primer to bead. Specifically, FIG. 15 shows on bead barcodes that comprise primers (barcodes) externally-linked to beads on the left hand side. On the right hand side, Figure shows template DNA and gene-specific primers (GSP).

An exemplary method according to one embodiment of the disclosure comprises: forming an affinity group on a solid substrate, wherein the solid substrate comprises a microsphere; forming a synthetic oligonucleotide having a read portion, a barcode and a common sequence; coupling the oligonucleotide to the affinity group of the barcode at a point of attachment. In an exemplary embodiment, the synthetic oligo may comprise a complementary sequence to the target sequence. In another embodiment, the affinity group may comprise acrylamide gel.

In an exemplary application, the disclosed embodiments provide a precision genomic platform enabled by a two-step microfluidic workflow and a high multiplex PCR biochemistry scheme. The two-step microfluidics allows for efficient access to DNA for downstream genomic reactions and provides flexibility to adapt for additional applications and multi-omics. The multiplex PCR chemistry (FIGS. 14, 15) can be developed and co-optimized with an AI-powered panel design pipeline and enables direct and efficient amplification of targeted genomic regions within barcoded individual cells. Taken together the platform produces high genomic coverage, low allele dropout rate, highly uniform amplification in thousands of cells from single run, is compatible with diverse and difficult samples, and is easily deployable for custom content.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method for detection of gene expression in a nucleic acid sample from a single cell, the method comprising:
selecting one or more target nucleic acid sequence in an individual cell, where the target nucleic acid sequence is contained in a DNA or RNA; providing a sample having one or more individual single cell;
encapsulating an individual cell in a drop;
incubating the encapsulated cell protease in the drop to produce a cell lysate;
providing a nucleic acid amplification primer set complementary to a target nucleic acid, where at least one primer of the nucleic acid amplification primer set comprises a barcode identification sequence;
performing a reverse transcription and nucleic acid amplification reaction to form an amplification product from the nucleic acid of a single cell; and
determining whether the target nucleic acid is expressed if the target nucleic acid comprises a transcript.

2. A method according to claim 1, further comprising:
providing an affinity reagent that comprises a nucleic acid sequence complementary to a barcode sequence of one of more nucleic acid primer, where the affinity reagent comprising said nucleic acid sequence complementary to the barcode sequence is capable of binding to a nucleic acid amplification primer comprising a barcode sequence; and
contacting an affinity reagent to the amplification product comprising amplicons under conditions sufficient for binding of the affinity reagent to the target nucleic acid to form an affinity reagent bound target nucleic acid.

3. A method according to claim 1, further comprising nucleic acid sequencing of an amplification product or amplicon to determine whether the target nucleic acid is present.

4. A method according to claim 1, further comprising nucleic acid sequencing of an amplification product or amplicon to determine the target nucleic acid level relative to other targeted nucleic acids.

5. A method according to claim 1, further comprising nucleic acid sequencing of an amplification product or amplicon to determine whether the target nucleic acid comprises a fusion transcript.

6. A method according to claim 1, further comprising nucleic acid sequencing of an amplification product or amplicon to determine whether the target nucleic acid comprises a BCR-ABL1 fusion transcript.

7. A method according to claim 1, wherein the nucleic acid amplification primer set spans a splice junction.

8. A method according to claim 1, wherein the nucleic acid amplification primer set spans a BCR-ABL splice junction.

9. A method according to claim 1, comprising a forward amplification primer having an embedded barcode identification sequence.

10. A method according to claim 1, comprising a reverse amplification primer having an embedded barcode identification sequence.

11. A method according to claim 1, wherein the method is used for the detection of an AML tumor.

12. A method according to claim 1, wherein the method is used for the detection of a leukemia.

13. A method according to claim 1, wherein the method is used for the detection of a myeloid leukemia.

14. A method according to claim 1, wherein the method is used to determine the prognosis of a patient suspected of having a BCR-ABL fusion transcript.

15. A method according to claim 1, wherein the nucleic acid amplification primer set comprises the sequence ACTCCAGACTGTCCACAGCA (SEQ ID NO: 1) or a variant thereof with one to three nucleotide substitutions or deletions at either end is used as a forward primer for amplification, and the sequence TTGGGGTCATTTTCACTGGGTCCAGCGAGAAGGT (SEQ ID NO: 2) or a variant thereof with one to four nucleotide substitutions or deletions at either end is used as a reverse primer for amplification.

16. A method according to claim 1, wherein an individual cell is encapsulated in a single drop comprising a reaction mixture in an aqueous, an aqueous emulsion in oil, or an aqueous suspension in oil.

17. A method according to claim 17, wherein the reaction mixture comprises proteinase K or another cytolytic protease.

18. A method according to claim 18, wherein the cytolytic protease is heat inactivated before or during the nucleic acid amplification reaction.

19. A method according to claim 1, wherein the nucleic acid amplification reaction is the polymerase chain reaction or a known variant thereof.

20. A BCR-ABL gene fusion.
